(19) United States
(12) Patent Application Publication
(10) Pub. No.: US 2015/0024488 A1
(43) Pub. Date: Jan. 22, 2015

(54) NUCLEIC ACID COMPLEX

(71) Applicants: Pathiraja Arachchillage Gunatillake, Mulgrave (AU); Carlos Gurrero-Sanchez, Dandenong (AU); Tracey Michelle Hinton, Belmont (AU); San Hoa Thang, Camberwell (AU); Mark Leslie Tizard, Highton (AU)

(72) Inventors: Pathiraja Arachchillage Gunatillake, Mulgrave (AU); Carlos Gurrero-Sanchez, Dandenong (AU); Tracey Michelle Hinton, Belmont (AU); San Hoa Thang, Camberwell (AU); Mark Leslie Tizard, Highton (AU)

(73) Assignee: COMMONWEALTH SCIENTIFIC AND INDUSTRIAL RESEARCH ORGANISATION, Australian Capital Territory (AU)

(21) Appl. No.: 14/129,249

(22) PCT Filed: Jun. 28, 2012
(86) PCT No.: PCT/AU2012/000759

§ 371 Date: May 1, 2014

(30) Foreign Application Priority Data
Jul. 4, 2011 (AU) 2011902654

(51) Int. Cl. A61K 48/00; C08F 293/00; C12N 15/113

(52) U.S. Cl. CPC A61K 48/0041; C12N 15/113; C08F 293/00
USPC 435/375; 525/54.2

(57) ABSTRACT

The present invention relates to a complex comprising a cationic block copolymer and a nucleic acid, the cationic block copolymer having at least a tri-block structure comprising a cationic block and two hydrophilic blocks, or a hydrophilic block and two cationic blocks.

### FIELD OF THE INVENTION

The present invention relates in general to nucleic acid complexes. More particularly, the invention relates to complexes of nucleic acids and cationic polymers, to the use of such complexes in methods of delivering a nucleic acid to a cell, and to a method of silencing gene expression. The invention further relates to the use of the cationic polymer in a method of protecting a nucleic acid from enzymatic degradation.

### BACKGROUND OF THE INVENTION

Considerable research effort has been directed toward developing techniques and agents for delivering nucleic acids to cells. For example, there has been considerable interest in developing delivery agents and techniques for delivering specific nucleic acid sequences that control gene expression and thus facilitate the treatment of conditions such as genetic diseases, viral infection and cancers.

Important parameters for successfully delivering nucleic acids to cells can include the use of an agent that forms a complex with the nucleic acid. The agent will typically be required to provide for a stable complex with the nucleic acid, possibly to protect the nucleic acid from enzymatic degradation, and/or facilitate transfection of the complexed nucleic acid.

A variety of agents have been developed for forming complexes with nucleic acids that facilitate delivery of the nucleic acids to cells. For example, lipid, calcium phosphate and cationic polymer agents have been successfully employed in forming nucleic acid complexes suitable for use in transfection methods. However, such agents and their use are subject to a number of limitations. For example, some agents are not compatible with a range of cell types. Furthermore, some agents are quite limited in terms of their ability to be designed/modified in order to tailor their use for forming a complex with different nucleic acids and/or for the resulting complex to be applicable for use with different cell types.

Accordingly, there remains an opportunity for developing complexes that can facilitate delivery of nucleic acids to cells, the likes of which offer improved function and/or an alternative to current complexes.

### SUMMARY OF THE INVENTION

The present invention therefore provides a complex comprising a cationic block copolymer and a nucleic acid, the cationic block copolymer having at least a tri-block structure comprising a cationic block and two hydrophilic blocks, or a hydrophilic block and two cationic blocks.

It has now been found that cationic block copolymers having at least a tri-block structure according to the present invention can form stable complexes with a variety of nucleic acids, with the resulting complex affording improved transfection for the nucleic acid to a variety of cell types. The cationic block copolymers when in the form of the complex have also been found to afford good protection to nucleic acids from enzymatic degradation.

Due to its block character, each block within the cationic block copolymer can advantageously be tailor designed to provide for efficient complexation with a given nucleic acid and/or for efficient transfection of the nucleic acid with a given cell type. The cationic block copolymer can also advantageously be tailor designed to incorporate a targeting ligand that directs the complex to a chosen targeted cell type.

Notably, cationic block copolymers having a tri-block structure used in accordance with the invention have been found to provide improved nucleic acid complex stability and transfection as compared with cationic block copolymers having a di-block structure.

In one embodiment, the at least tri-block structure of the cationic block copolymer is linear and comprises a cationic block and two hydrophilic blocks where the cationic block is located in between each of the two hydrophilic blocks.

In another embodiment, the at least tri-block structure of the cationic block copolymer is linear and comprises a hydrophilic block and two cationic blocks where the hydrophilic block is located in between each of the two cationic blocks.

In a further embodiment, the at least tri-block structure of the cationic copolymer is linear and comprises a cationic block and two hydrophilic blocks where the cationic block is located in between and directly coupled to each of the two hydrophilic blocks. In that case, the tri-block structure of the cationic block copolymer may be conveniently referred to as having an A-B-A tri-block structure, where each A may be the same or different and represents a hydrophilic block, and B represents the cationic block.

In yet a further embodiment, the at least tri-block structure of the cationic copolymer is linear and comprises a hydrophilic block and two cationic blocks where the hydrophilic block is located in between and directly coupled to each of the two cationic blocks. In that case, the tri-block structure of the cationic block copolymer may be conveniently referred to as having a B-A-B tri-block structure, where each B may be the same or different and represents a cationic block, and A represents the hydrophilic block.

The present invention also provides a method of delivering a nucleic acid to a cell, the method comprising:

preparing a complex comprising a cationic block copolymer and a nucleic acid, the cationic block copolymer having at least a tri-block structure comprising a cationic block and two hydrophilic blocks, or a hydrophilic block and two cationic blocks; and

introducing the complex to the cell.

In one embodiment, the nucleic acid is delivered to a cell for the purpose of silencing gene expression.

The present invention therefore also provides a method of silencing gene expression, the method comprising transfecting a cell with a complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

In one embodiment, the DNA and RNA are selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear (SnRNAs) ribozymes, aptamers, DNAzymes, ribonuclease-type complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

Cationic block copolymers used in accordance with the invention have also been found to impart to nucleic acids protection against enzymatic degradation.

The present invention therefore also provides a method of protecting a nucleic acid form enzymatic degradation, the method comprising complexing the nucleic acid with a cationic block copolymer, the cationic block copolymer having at least a tri-block structure comprising a cationic block and two hydrophilic blocks, or a hydrophilic block and two cationic blocks.

There is also provided use of a complex for delivering a nucleic acid to a cell, the complex comprising a cationic block copolymer and the nucleic acid, the cationic block copolymer having at least a tri-block structure comprising a cationic block and two hydrophilic blocks, or a hydrophilic block and two cationic blocks.

There is further provided use of a complex in the manufacture of a composition for delivering a nucleic acid to a cell, the complex comprising a cationic block copolymer and the nucleic acid, the cationic block copolymer having at least a tri-block structure comprising a cationic block and two hydrophilic blocks, or a hydrophilic block and two cationic blocks.

The present invention also provides use of a complex for silencing gene expression, the complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

The present invention further provides use of a complex in the manufacture of a composition for silencing gene expression, the complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

In one embodiment, the DNA and RNA are selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear (SnRNAs) ribozymes, aptamers, DNAzymes, ribonuclease-type complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

The present invention also provides use of a cationic block copolymer in protecting a nucleic acid from enzymatic degradation, the cationic block copolymer having at least a tri-block structure comprising a cationic block and two hydrophilic blocks, or a hydrophilic block and two cationic blocks.

The present invention further provides use of a cationic block copolymer in the manufacture of a composition for protecting a nucleic acid from enzymatic degradation, the cationic block copolymer having at least a tri-block structure comprising a cationic block and two hydrophilic blocks, or a hydrophilic block and two cationic blocks.

Further aspects of the invention appear below and the detailed description of the invention.

### BRIEF DESCRIPTION OF THE DRAWINGS

The invention will herein be described with reference to the following non-limiting drawings in which:

FIG. 1 illustrates viability of CHO-GFP and HEK293T cells exposed to ABA tri block copolymers prepared in Example 1;

FIG. 2 illustrates association of tri-block copolymer with siRNA as a function of polymer:siRNA ratio (w/w) for the series of polymers prepared in Example 1. Also shown is the corresponding N/P ratio;

FIG. 3 illustrates gene silencing in CHO-GFP cells for different siRNA: RAFT polymer (prepared in Example 1) combinations presented as a percentage of L2000 diRNA samples or polymer/diRNA complexes mean EGFP (measured on FITC wavelength) fluorescence;

FIG. 4 illustrates gene silencing in CHO-GFP cells for different siRNA:422-3 polymer (prepared in Example 1) concentrations presented as a percentage of non-silencing siRNA for L2000 samples or polymer/diRNA complexes mean EGFP (measured on FITC wavelength) fluorescence;

FIG. 5 illustrates stability of siRNA/422-3 polymer complex in foetal bovine serum (FBS); (A) stability of naked siRNA, (B) 1007-2:siRNA 4:1, (C) 1007-2:siRNA 4:1, (D) 422-3:siRNA 4:1 and (E) ability of the treated complexes to silence in CHO-GFP cells;

FIG. 6 illustrates the cell viability of triblock copolymers prepared in Example 6 (a) and diblock copolymers prepared in Example 7 (b);

FIG. 7 illustrates results of electrophoresis tests to demonstrate the siRNA uptake of block copolymers prepared in Examples 6 and 7, where JG20A, JG20B, JG20C, JG20D, CG408A, CG408B is a reference to 189JG20A, 189JG20B, 189JG20C, 189JG20D, 0408-A, 0408-B, respectively;

FIG. 8 illustrates relative silencing efficiency of triblock copolymers (Example 6) and diblock copolymers (Example 7) as measured by fluorescence using plate reader (a) and FACS (b and c);

FIG. 9 illustrates binding of 1007-2 (unlabelled) vs 1007-2/PF (labeled) as demonstrated by electrophoresis;

FIG. 10 illustrates silencing of CHO-GFP by labeled (1007-2 PF) and unlabelled (1007-2) RAFT polymer;

FIG. 11 illustrates cellular uptake of RAFT polymer particles by CHO and Huh-GFP cells: polymer was added 2 hours prior to fixation of cells. Polymer signal is red, DAPI stains the nucleus (blue), GFP (green) outlines the cells;

FIG. 12 illustrates uptake of 1007-2/PF (prepared in Example 10) and si22 complexes in Chicken Embryos at 6 h (A) and 24 h (B). Polymer±si22 was injected into the allantoic fluid of day 10 embryonated chicken eggs and incubated at 37° C. for 6 or 24 h. Allantoic membrane was removed and fixed in 4% paraformaldehyde for 2 h. Membranes were then permeabilized for 1 h in 0.1% Triton X-100, and stained with DAPI for 20 min to visualize nuclei;

FIG. 13 illustrates toxicity of 1007-2 in chicken embryos (A & B) IFN response to 1007-2 in Chicken Embryos. Polymer±si22 was injected into the allantoic fluid of day 10 embryonated chicken eggs and incubated at 37° C. for 6 or 24 h. Allantoic membrane was removed and total RNA was purified and subjected to qRT-PCR for IFNα and β compared to GAPDH. Results represent 5 chicken embryos per group±SEM. Statistics *P<0.05 compared to PBS. One way repeated measures ANOVA were performed with a parametric Tukey post analysis (C, D & E);

FIG. 14 illustrates influenza virus inhibition in chicken embryos. Polymer±relevant siRNAs were injected into the allantoic fluid of day 10 embryonated chicken eggs and incubated for 24 h. 500 pfu of PR8 was injected into the allantoic fluid of each embryo and incubated at 37° C. for a further 48 h. Allantoic fluid was harvested and TCID₅₀'s performed. Results represent 5 chicken embryos per group±SEM. Statistics *P<0.05 compared to PBS Δ<0.05 compared to 1007-2/si22. One way repeated measures ANOVA, parametric, Tukey post analysis;

FIG. 15 illustrates silencing of CHO-GFP by RAFT polymers containing boronic acid (sample BC6-1) and RAFT polymers with galactose complexed to boronic acid moieties (BC14);

FIG. 16 illustrates siRNA binding with three ABA tri-block copolymers polymers with different block copolymer lengths as demonstrated by electrophorosis. The Figure also illustrates the binding of siRNA at different molar ratios with each polymer; and

FIG. 17 illustrates cell viability (top panel) of siRNA and polymer complexes at different molar ratios (N:P) and the CHO-GFP silencing (bottom panel).

Some Figures contain colour representations or entities. Coloured versions of the Figures are available upon request.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

As used herein, the singular forms “a”, “and” and “the” are intended to include plural aspects unless the context clearly dictates otherwise. Thus, for example, reference to “a cell” includes a single cell as well as two or more cells; reference to “an agent” includes one agent, as well as two or more agents; and so forth.

The present invention provides a complex comprising a cationic block copolymer and a nucleic acid. The term “complex” as used herein refers to the association by ionic bonding of the cationic block copolymer and the nucleic acid. The ionic bonding is derived through electrostatic attraction between oppositely charged ions associated with the cationic block copolymer and the nucleic acid. It will be appreciated that the cationic block copolymer will provide for positive charge, and accordingly the nucleic acid will provide for negative charge.

There is no particular limitation concerning the ratio of cationic block copolymer to nucleic acid that may be used to form the complex. In one embodiment, the molar ratio of cationic block copolymer to nucleic acid ranges from 1:1 to 15:1, or from 1:1 to 10:1. or from 2:1 to 10:1, or from 3:1 to 10:1, or from 4:1 to 10:1. In another embodiment, the molar ratio of cationic block copolymer to nucleic acid ranges from 2:1 to 7:1.

Those skilled in the art will appreciate that charge density (as indicated by Zeta potential) of the cationic block copolymer and nucleic acid, together with the ratio of cationic block copolymer to nucleic acid, will effect the overall charge/neutral state of the resulting complex.

In one embodiment, the complex has a positive Zeta potential. In a further embodiment, the complex has a positive Zeta potential ranging from greater than 0 mV to about 50 mV, for example from about 4 mV, 5 mV, 6 mV, 7 mV, 8 mV, 9 mV, or 10 mV to about 40 mV, or from about 10 mV to about 40 mV, or from about 15 mV to about 30 mV, or from about 20 mV to about 30 mV.

The Zeta potential of a complex in accordance with the present invention is that as measured by Malvern Zetasizer. The Zeta potential is calculated from the measurement of the mobility of particles (electrophoertic mobility) in an electrical field and the particle size distribution in the sample.

The term “nucleic acid” used herein refers to nucleic acid molecules including DNA (gDNA, cDNA), oligonucleotides (double or single stranded), RNA (sense RNAs, antisense RNAs, mRNAs, tRNAs, rRNAs, small interfering RNAs (siRNAs), double-stranded RNAs (dsRNA), short hairpin RNAs (shRNAs), piwi-interacting RNAs (PiRNA), micro RNAs (miRNAs), small nucleolar RNAs (SnoRNAs), small nuclear RNAs (SnRNAs)), ribozymes, aptamers, DNAzymes, ribonuclease-type complexes and other such molecules as herein described. For the avoidance of doubt, the term “nucleic acid” includes non-naturally occurring modified forms, as well as naturally occurring forms.

In some embodiments, the nucleic acid molecule comprises from about 8 to about 80 nucleobases (i.e. from about 8 to about 80 consecutively linked nucleic acids). One of ordinary skill in the art will appreciate that the present invention embodies nucleic acid molecules of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

The term “nucleic acid” also includes other families of compounds such as oligonucleotide analogs, chimeric, hybrid and mimetic forms.

Chimeric oligomeric compounds may also be formed as composite structures of two or more nucleic acid molecules, including, but not limited to, oligonucleotides, oligonucleotide analogs, oligonucleosides and oligonucleotide mimetics. Routinely used chimeric compounds include but are not limited to hybrids, hemimers, gapmers, extended gapmers, inverted gapmers and blockmers, wherein the various point modifications and or regions are selected from native or modified DNA and RNA type units and/or mimetic type subunits such as, for example, locked nucleic acids (LNA), peptide nucleic acids (PNA), morpholinos, and others. The preparation of such hybrid structures is described for example in U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

RNA and DNA aptamers are also contemplated. Aptamers are nucleic acid molecules having specific binding affinity to non-nucleic acid or nucleic acid molecules through interactions other than classic Watson-Crick base pairing. Aptamers are described, for example, in U.S. Pat. Nos. 5,475,096; 5,270,163; 5,589,332; 5,589,332; and 5,741,679. An increasing number of DNA and RNA aptamers that recognize their non-nucleic acid targets have been developed and have been characterized (see, for example, Gold et al., Annu. Rev. Biochem., 64: 763-797.1995; Bacher et al., Drug Discovery Today, 3(6): 265-273, 1998).

Further modifications can be made to the nucleic acid molecules and may include conjugate groups attached to one of the termini, selected nucleobase positions, sugar positions or to one of the internucleoside linkages.

The cationic block copolymer used in accordance with the invention has at least a tri-block structure comprising a cationic block and two hydrophilic blocks, or a hydrophilic block and two cationic blocks. In its simplest form, the cationic block copolymer may be a tri-block copolymer. However, the tri-block copolymer may form part of a higher block copolymer, such as a tetra-, penta-, or a hexa- etc block copolymer.

By the cationic block copolymer comprising a “cationic block” is meant a discernable block within the copolymer structure that presents a net positive charge.

By the cationic block copolymer comprising a “hydrophilic block” is meant a discernable block within the copolymer structure that presents net hydrophilic character.

In one embodiment, the at least tri-block structure of the cationic block copolymer is linear and comprises a cationic block and two hydrophilic blocks where the cationic block is located in between each of the two hydrophilic blocks.

In another embodiment, the at least tri-block structure of the cationic block copolymer is linear and comprises a hydrophilic block and two cationic blocks where the hydrophilic block is located in between each of the two cationic blocks.

In a further embodiment, the at least tri-block structure of the cationic copolymer is linear and comprises a cationic block and two hydrophilic blocks where the cationic block is located in between and directly coupled to each of the two hydrophilic blocks. In that case, the tri-block structure of the cationic block copolymer may be conveniently referred to as having an A-B-A tri-block structure, where each A may be the same or different and represents a hydrophilic block, and B represents the cationic block.

In yet a further embodiment, the at least tri-block structure of the cationic copolymer is linear and comprises a hydrophilic block and two cationic blocks where the hydrophilic block is located in between and directly coupled to each of the two cationic blocks. In that case, the tri-block structure of the cationic block copolymer may be conveniently referred to as having a B-A-B tri-block structure, where each B may be the same or different and represents a cationic block, and A represents the hydrophilic block.

Where present, each of the two cationic blocks or each of the two hydrophilic blocks may be the same or different.

Each block in the tri-block structure of the cationic block copolymer may be a homopolymer block or a copolymer block. Where a block of the tri-block structure is a copolymer, the copolymer may be a gradient copolymer or a random or statistical copolymer.

Depending upon how the cationic block copolymer is prepared, it may be that at least one block within the tri-block structure comprises a linking group, which may be, for example, a residue of a moiety that facilitates polymerisation of monomer to form the cationic block copolymer. In that case, the tri-block structure of the cationic block copolymer may, for example, be represented as A-B-L-B-A, where each A independently represents the two hydrophilic blocks and B-L-B represents the cationic block.

Again, depending upon how the cationic block copolymer is prepared, it may be that one or both of the two hydrophilic blocks (or one or both of the two cationic blocks) comprise a terminal residue of a moiety used to facilitate polymerisation of monomer to form the cationic block copolymer. For example, the tri-block structure of the cationic block copolymer may be represented as X-A-B-L-B-A-X, where A, B and L are as defined directly above and X is a residue of a moiety used to facilitate polymerisation of monomer to form the cationic block copolymer. Each X may be the same or different.

By being a residue of a moiety used to facilitate polymerisation of monomer to form the cationic block copolymer, X and L will generally not be polymeric in their own right. Despite the presence of such X and L residues within the tri-block structure, those skilled in the art will appreciate that the structure “-B-L-B-” will be considered equivalent to the block “-B-”. Similarly, the structure “X-A-” will be considered equivalent to the block “A-”. For example, where a reversible addition fragmentation chain transfer (RAFT) agent is used to facilitate polymerisation of monomer to form the cationic block copolymer, the cationic or hydrophilic block(s) may comprise a residue of the RAFT agent. This is illustrated below in Schematic 1 by way of reference to an exemplar RAFT agent that may be used to form a cationic block copolymer for use in accordance with the invention.

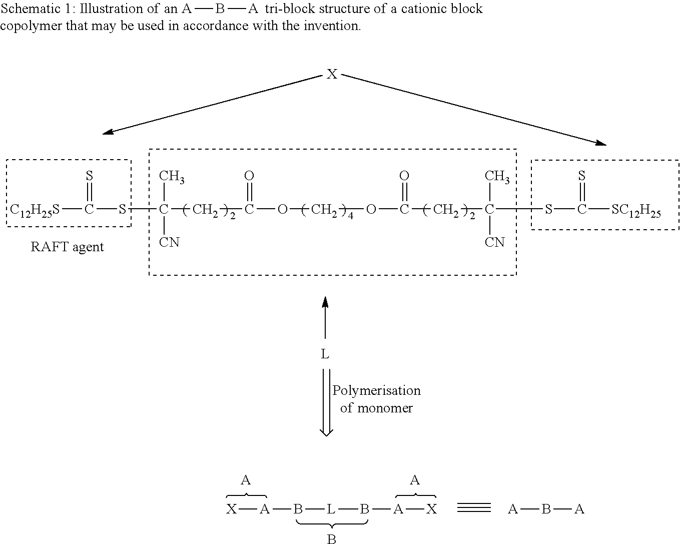

With reference to Schematic 1, the specific RAFT agent illustrated can be seen to comprise components X (×2) and L. Upon the RAFT agent being used to facilitate polymerisation of monomer to form the cationic block and each of the two hydrophilic blocks, the resulting cationic block copolymer can be seen to comprise components X-A (×2) and B-L-B, which in turn is considered equivalent to the tri-block structure A-B-A, where A and B are as herein defined.

The hydrophilic block(s) and the cationic block(s) will generally comprise the polymerised residues of a plurality of monomer units (i.e. polymerised monomer residue units). The polymerised monomer residue units that make up the hydrophilic block(s) and the cationic block(s) can also be referred to in the art as monomer repeat units or simply as repeart units. Further detail concerning the monomers that may be used to form the blocks is outlined below.

A cationic block may comprise from about 5 to about 200, or about 40 to about 200, or about 80 to about 200 polymerised monomer residue units. Where the cationic block copolymer comprises two cationic blocks, each cationic block may independently comprise from about 5 to about 100, or about 20 to about 100, or about 40 to about 100 polymerised monomer residue units. Individually or collectively, the cationic block(s) will present a net positive charge. Generally at least about 10%, or at least 30%, or at least 40%, or at least 50%, or at least 70%, or at least 90%, or all of the polymerised monomer residue units that make up the cationic block comprise a positive charge.

In one embodiment, a cationic block comprises from about 5 to about 200, or about 40 to about 200, or about 80 to about 200 polymerised monomer residue units that each comprise positive charge.

Where the cationic block copolymer comprises two cationic blocks, each cationic block may independently comprise from about 5 to about 100, or about 20 to about 100, or about 40 to about 100 polymerised monomer residue units that each comprise positive charge.

It will be appreciated that individually or collectively the cationic block(s) will comprise sufficient positive charge density to promote complexation with the nucleic acid.

A hydrophilic block may comprise from about 5 to about 200, or from about 30 to about 200, or from about 40 to about 180, or from about 50 to about 180, or from about 60 to about 180 polymerised monomer residue units. Where the cationic block copolymer comprises two hydrophilic blocks, each hydrophilic block may independently comprise from about 5 to about 100, or about 15 to about 100, or about 20 to about 90, or from about 25 to about 90, or from about 30 to about 90 hydrophilic polymerised monomer residue units. Individually or collectively, the hydrophilic block(s) will present net hydrophilic character.

Generally, at least about 50%, or at least about 60%, or at least about 70%, or at least about 90%, or about 100% of the polymerised monomer residue units that form a hydrophilic block will be hydrophilic monomer residue units.

In one embodiment, a hydrophilic block comprises from about 5 to about 200, or from about 30 to about 200, or from about 40 to about 180, or from about 50 to about 180, or from about 60 to about 180 hydrophilic polymerised monomer residue units.

Where the cationic block copolymer comprises two hydrophilic blocks, each hydrophilic block may independently comprise from about 5 to about 100, or about 15 to about 100, or about 20 to about 90, or from about 25 to about 90, or from about 30 to about 90 hydrophilic polymerised monomer residue units.

Terms such as hydrophilic and hydrophobic are generally used in the art to convey interactions between one component relative to another (e.g. attractive or repulsive interactions, or solubility characteristics) and not to quantitatively define properties of a particular component relative to another.

For example, a hydrophilic component is more likely to be wetted or solvated by an aqueous medium such as water, whereas a hydrophobic component is less likely to be wetted or solvated by an aqueous medium such as water.

In the context of the present invention, a hydrophilic block is intended to mean a polymer block that exhibits solubility in an aqueous medium, including biological fluids such as blood, plasma, serum, urine, saliva, milk, seminal fluid, vaginal fluid, synovial fluid, lymph fluid, amniotic fluid, sweat, and tears; as well as an aqueous solution produced by a plant, including, for example, exudates and guttation fluid, xylem, phloem, resin, and nectar.

The hydrophilic block(s) will generally be selected such that the resulting cationic block copolymer is rendered soluble in aqueous media.

The cationic block(s) may also exhibit hydrophilic properties such that it is soluble in aqueous media.

The cationic block copolymer will generally not comprise monomer residue units' bearing negative charge. In other words, the cationic block copolymer will generally not be an ampholytic polymer.

Reference herein to “positive” or “negative” charge is intended to mean that a moiety or functional group of the block copolymer or nucleic acid presents a positive or negative charge, respectively. The moiety or functional group may of course initially be in a neutral state and subsequently be converted into a charged state. Thus, the functional group or moiety may inherently bear charge, or it may be capable of being converted into a charged state, for example through addition or removal of an electrophile. In other words, in the case of a positive charge, the functional group or moiety may have an inherent charge such as a quaternary ammonium functional group or moiety, or the functional group or moiety per se may be neutral, yet be chargeable to form a cation through, for example, pH dependent formation of a tertiary ammonium cation, or quaternerisation of a tertiary amine group. In the case of negative charge, the functional group or moiety may, for example, comprise an organic acid salt that provides for the negative charge, or the functional group or moiety may comprise an organic acid which may be neutral, yet be chargeable to form an anion through, for example, pH dependent removal of an acidic proton.

In one embodiment, the cationic block may be prepared using monomer that contains a functional group or moiety that is in a neutral state and can subsequently converted into a positively charged state. For example, the monomer may comprise a tertiary amine functional group, which upon being polymerised to form the cationic block is quaternarised into a positively charged state.

Those skilled in the art will appreciate that in a charged state, a cation associated with the cationic block copolymer per se, or an anion associated with the nucleic acid per se will have a suitable counter ion associated with it.

In order to form the complex in accordance with the invention, the cationic block(s) must of course comprise positive charge and the nucleic acid must of course comprise negative charge so as to promote electrostatic attraction and formation of the complex.

The net negative charge on the nucleic acid molecule will generally be derived from the negatively charged nucleic acids per se (e.g. from the phosphate groups).

The cationic block copolymer provides for positive charge, and accordingly the nucleic acid will provide for negative charge. Thus, it would be understood that any modification(s) made to the nucleic acid molecule should retain a net negative charge to the extent that it allows formation of a complex through ionic bonding with the cationic block copolymer.

The complex comprising the cationic block copolymer and nucleic acid may be prepared using known techniques for preparing cationic polymer/nucleic acid complexes. For example, a required amount of polymer suspended in water may be introduced to a container comprising reduced serum media such as Opti-MEM®. The required amount of nucleic acid may then be introduced to this solution and the resulting mixture vortexed for an appropriate amount of time so as to form the complex.

The nucleic acid may be obtained commercially or prepared or isolated using techniques well known in the art.

The cationic block copolymer may be prepared by any suitable means.

In one embodiment, the cationic block copolymer is prepared by polymerisation of ethylenically unsaturated monomers. Polymerisation of the ethylenically unsaturated monomers is preferably conducted using a living polymerisation technique.

Living polymerisation is generally considered in the art to be a form of chain polymerisation in which irreversible chain termination is substantially absent. An important feature of living polymerisation is that polymer chains will continue to grow while monomer and reaction conditions to support polymerisation are provided. Polymer chains prepared by living polymerisation can advantageously exhibit a well defined molecular architecture, a predetermined molecular weight and narrow molecular weight distribution or low polydispersity.

Examples of living polymerisation include ionic polymerisation and controlled radical polymerisation (CRP). Examples of CRP include, but are not limited to, iniferter polymerisation, stable free radical mediated polymerisation (SFRP), atom transfer radical polymerisation (ATRP), and reversible addition fragmentation chain transfer (RAFT) polymerisation.

Equipment, conditions, and reagents for performing living polymerisation are well known to those skilled in the art.

Where ethylenically unsaturated monomers are to be polymerised by a living polymerisation technique, it will generally be necessary to make use of a so-called living polymerisation agent. By “living polymerisation agent” is meant a compound that can participate in and control or mediate the living polymerisation of one or more ethylenically unsaturated monomers so as to form a living polymer chain (i.e. a polymer chain that has been formed according to a living polymerisation technique).

Living polymerisation agents include, but are not limited to, those which promote a living polymerisation technique selected from ionic polymerisation and CRP.

In one embodiment of the invention, the cationic block copolymer is prepared by ionic polymerisation.

Living ionic polymerisation is a form of addition polymerisation whereby the kinetic-chain carriers are ions or ion pairs. The polymerisation proceeds via anionic or cationic kinetic-chain carriers. In other words, the propagating species will either carry a negative or positive charge, and as such there will also be an associated counter cation or counter anion, respectively. For example, in the case of anionic polymerisation, the living polymerisation agent might be represented as I⁻M⁺, where I represents an organo-anion (e.g. an optionally substituted alkyl anion) and M represents an associated countercation, or in the case of living cationic polymerisation, the living polymerisation agent might be represented as I⁺M⁻, where I represents an organo-cation (e.g. an optionally substituted alkyl cation) and M represents an associated counteranion. Suitable agents for conducting anionic and cationic living polymerisation are well known to those skilled in the art and include, but are not limited to, aprotonic acids (e.g. aluminium trichloride, boron trifluoride), protonic (Bronstead) acids, stable carbenium-ion salts, organometallic compounds (e.g. N-butyl lithium, cumyl, potassium) and Ziegler-Natta catalysts (e.g. triethyl aluminium and titanium tetrachloride).

In one embodiment of the invention, the cationic block copolymer is prepared by CRP.

In a further embodiment of the invention, the cationic block copolymer is prepared by iniferter polymerisation.

Iniferter polymerisation is a well known form of CRP, and is generally understood to proceed by a mechanism illustrated below in Scheme 2.

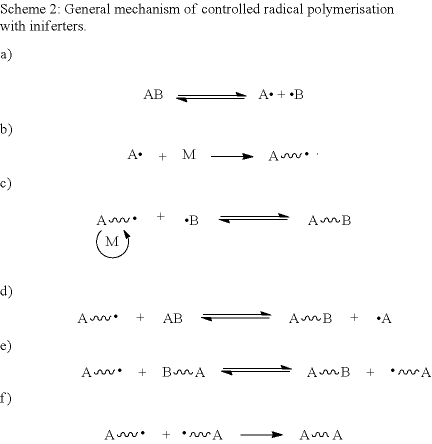

With reference to Scheme 2, the iniferter agent AB dissociates chemically, thermally or photochemically to produce a reactive radical species A and generally a relatively stable radical species B (for symmetrical iniferters the radical species B will be the same as the radical species A) (step a). The radical species A can initiate polymerisation of monomer M (in step b) and may be deactivated by coupling with radical species B (in step c). Transfer to the iniferter (in step d) and/or transfer to dormant polymer (in step e) followed by termination (in step d) characterise iniferter chemistry. Suitable iniferter agents are well known to those skilled in the art, and include, but are not limited to, dithiocarbonate, disulphide, and thiuram disulphide compounds.

In a further embodiment of the invention, the cationic block copolymer is prepared by SFRP.

As suggested by its name, this mode of radical polymerisation involves the generation of a stable radical species as illustrated below in Scheme 3.

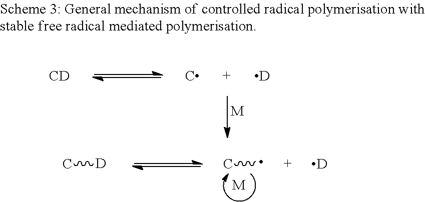

With reference to Scheme 3, SFRP agent CD dissociates to produce an active radical species C and a stable radical species D. The active radical species C reacts with monomer M, which resulting propagating chain may recombine with the stable radical species D. Unlike iniferter agents, SFRP agents do not provide for a transfer step. Suitable agents for conducting SFRP are well known to those skilled in the art, and include, but are not limited to, moieties capable of generating phenoxy and nitroxy radicals. Where the agent generates a nitroxy radical, the polymerisation technique is more commonly known as nitroxide mediated polymerisation (NMP).

Examples of SFRP agents capable of generating phenoxy radicals include those comprising a phenoxy group substituted in the 2 and 6 positions by bulky groups such as tert-alkyl (e.g. t-butyl), phenyl or dimethylbenzyl, and optionally substituted at the 4 position by an alkyl, alkyloxy, aryl, or aryloxy group or by a heteroatom containing group (e.g. S, N or O) such as dimethylamino or diphenylamino group. Thiophenoxy analogues of such phenoxy containing agents are also contemplated.

SFRP agents capable of generating nitroxy radicals include those comprising the substituent R¹R²N—O—, where R¹ and R² are tertiary alkyl groups, or where R¹ and R² together with the N atom form a cyclic structure, preferably having tertiary branching at the positions α to the N atom. Examples of such nitroxy substituents include 2,2,5,5-tetraalkylpyrrolidinoxyl, as well as those in which the 5-membered hetrocycle ring is fused to an alicyclic or aromatic ring, hindered aliphatic dialkylaminoxyl and iminoxyl substituents. A common nitroxy substituent employed in SFRP is 2,2,6,6-tetramethyl-1-piperidinyloxy.

In another embodiment of the invention, the cationic block copolymer is prepared by ATRP.

ATRP generally employs a transition metal catalyst to reversibly deactivate a propagating radical by transfer of a transferable atom or group such as a halogen atom to the propagating polymer chain, thereby reducing the oxidation state of the metal catalyst as illustrated below in Scheme 4.

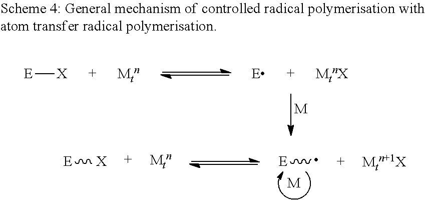

With reference to Scheme 4, a transferable group or atom (X, e.g. halide, cyanato, thiocyanato or azido) is transferred from the organic compound (E-X) to a transition metal catalyst (Mₜ, e.g. copper, iron, palladium, cobalt, rhenium, rhodium, ruthenium, molybdenum, niobium, or nickel) having oxidation number (n), upon which a radical species is formed that initiates polymerisation with monomer (M). As part of this process, the metal complex is oxidised (Mₜⁿ⁺¹X). A similar reaction sequence is then established between the propagating polymer chain and the dormant X end-capped polymer chains.

In a further embodiment of the invention, the cationic block copolymer is prepared by RAFT polymerisation.

RAFT polymerisation is well known in the art and is believed to operate through the mechanism outlined below in Scheme 5.

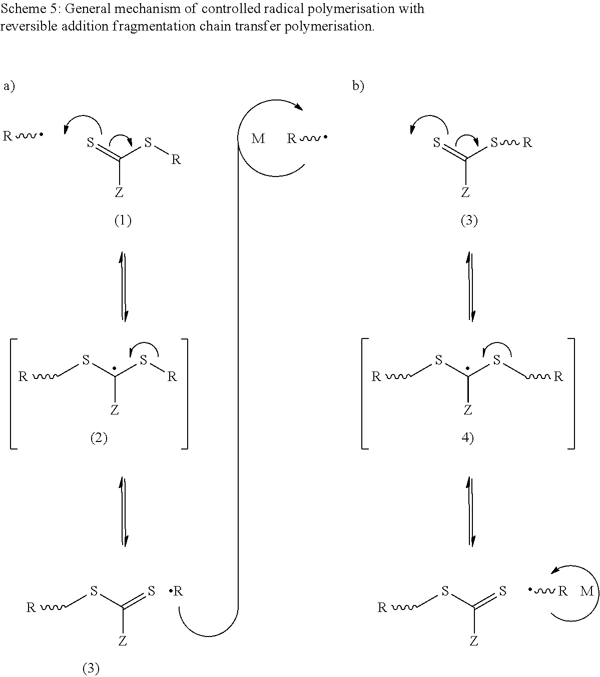

With reference to Scheme 5, RAFT polymerisation is believed to proceed through initial reaction sequence (a) that involves reaction of a RAFT agent (1) with a propagating radical. The labile intermediate radical species (2) that is formed fragments to form a temporarily deactivated dormant polymer species (3) together a radical (R) derived from the RAFT agent. This radical can then promote polymerisation of monomer (M), thereby reinitiating polymerisation. The propagating polymer chain can then react with the dormant polymer species (3) to promote the reaction sequence (b) that is similar to reaction sequence (a). Thus, a labile intermediate radical (4) is formed and subsequently fragments to form again a dormant polymer species together with a radical which is capable of further chain growth.

A polymer formed by RAFT polymerisation may conveniently be referred to as a RAFT polymer. By virtue of the mechanism of polymerisation, such polymers will comprise residue of the RAFT agent that facilitated polymerisation of the monomer.

RAFT agents suitable for use in accordance with the invention comprise a thiocarbonylthio group (which is a divalent moiety represented by: —C(S)S—). Examples of RAFT agents are described in Moad G.; Rizzardo, E; Thang S, H. Polymer 2008, 49, 1079-1131 and Aust. J. Chem., 2005, 58, 379-410; Aust. J. Chem., 2006, 59, 669-692; Aust. J. Chem., 2009, 62, 1402-1472 (the entire contents of which are incorporated herein by reference) and include xanthate, dithioester, dithiocarbamate and trithiocarbonate compounds.

A RAFT agent suitable for use in accordance with the invention may be represented by general formula (I) or (II):

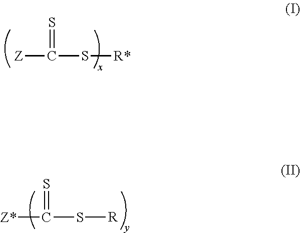

where Z and R are groups, and R* and Z* are x-valent and y-valent groups, respectively, that are independently selected such that the agent can function as a RAFT agent in the polymerisation of one or more ethylenically unsaturated monomers; x is an integer ≧1; and y is an integer ≧2.

In order to function as a RAFT agent in the polymerisation of one or more ethylenically unsaturated monomers, those skilled in the art will appreciate that R and R* will typically be an optionally substituted organic group that function as a free radical leaving group under the polymerisation conditions employed and yet, as a free radical leaving group, retain the ability to reinitiate polymerisation. Those skilled in the art will also appreciate that Z and Z* will typically be an optionally substituted organic group that function to give a suitably high reactivity of the C═S moiety in the RAFT agent towards free radical addition without slowing the rate of fragmentation of the RAFT-adduct radical to the extent that polymerisation is unduly retarded.

In formula (I), R* is a x-valent group, with x being an integer ≧1. Accordingly, R* may be mono-valent, di-valent, tri-valent or of higher valency. For example, R* may be a C₂₀ alkyl chain, with the remainder of the RAFT agent depicted in formula (I) presented as multiple substituent groups pendant from the chain. Generally, x will be an integer ranging from 1 to about 20, for example from about 2 to about 10, or from 1 to about 5. In one embodiment, x=2.

Similarly, in formula (II), Z* is a y-valent group, with y being an integer ≧2. Accordingly, Z* may be di-valent, tri-valent or of higher valency. Generally, y will be an integer ranging from 2 to about 20, for example from about 2 to about 10, or from 2 to about 5.

Examples of R in RAFT agents used in accordance with the invention include optionally substituted, and in the case of R* in RAFT agents used in accordance with the invention include a x-valent form of optionally substituted, alkyl, alkenyl, alkynyl, aryl, acyl, carbocyclyl, heterocyclyl, heteroaryl, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkylcarbocyclyloxy, alkylheterocyclyloxy, alkylheteroaryloxy, alkylthioalkyl, alkenylthioalkyl, alkynylthioalkyl, arylthioalkyl, alkylacylthio, alkylcarbocyclylthio, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, alkylthioaryl, alkenylthioaryl, alkynylthioaryl, arylthioaryl, arylacylthio, arylcarbocyclylthio, arylheterocyclylthio, arylheteroarylthio, and a polymer chain.

For avoidance of any doubt reference herein to “optionally substituted”, alkyl, alkenyl etc, is intended to mean each group such as alkyl and alkenyl is optionally substituted.

Examples of R in RAFT agents used in accordance with the invention also include optionally substituted, and in the case of R* in RAFT agents used in accordance with the invention also include an x-valent form of optionally substituted, alkyl; saturated, unsaturated or aromatic carbocyclic or heterocyclic ring; alkylthio; dialkylamino; an organometallic species; and a polymer chain.

More specific examples of R in RAFT agents used in accordance with the invention include optionally substituted, and in the case of R* in RAFT agents used in accordance with the invention include an x-valent form of optionally substituted, C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl, C₇-C₂₄ alkylaryl, C₂-C₁₈ alkylaryl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂-C₁₈ alkylthioalkyl, C₃-C₁₈ alkenylthioalkyl, C₃-C₁₈ alkynylthioalkyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, and a polymer chain having a number average molecular weight in the range of about 500 to about 80,000, for example in the range of about 500 to about 30,000.

More specific examples of a polymer chain include polystyrene, polyacrylamide, poly(methyl acrylate), poly(methyl methacrylate), poly(n-butyl acrylate), poly (tert-butyl acrylate), poly(acrylic acid), poly (vinyl acetate), poly(vinyl pyrrolidone), poly(N-isopropyl acrylamide), polystyrene-block-poly(tert-butyl acrylate), polystyrene-block-poly(acrylic acid), poly (para-acetoxystryene), poly(para-hydroxystyrene), poly(N,N-dimethyl acrylamide, poly(hydroxyethyl acrylate), poly(oligoethylene glycol acrylate), poly(N,N-dimethylaminoethyl methacrylate), poly(N-acryloylmorpholine), poly(methyl methacrylate)-block-poly(styrene), poly(ethyleneoxide)-block-poly(methyl methacrylate, poly(ethyleneoxide)-block-poly(N-isopropyl acrylamide), and poly(ethyleneoxide)-block-polystyrene-block-poly(acrylic acid).

Where R in RAFT agents used in accordance with the invention include, and in the case of R* in RAFT agents used in accordance with the invention include an x-valent form of, an optionally substituted polymer chain, the polymers chain may be formed by any suitable polymerisation process such as radical, ionic, coordination, step-growth or condensation polymerisation.

Living polymerisation agents that comprise a polymer chain are commonly referred to in the art as “macro” living polymerisation agents. Such “macro” living polymerisation agents may conveniently be prepared by polymerising one or more ethylenically unsaturated monomers under the control of a given living polymerisation agent.

In one embodiment, the polymer chain is formed by polymerising ethylenically unsaturated monomer under the control of a RAFT agent.

Examples of Z in RAFT agents used in accordance with the invention include optionally substituted, and in the case of Z* in RAFT agents used in accordance with the invention include a y-valent form of optionally substituted: F, Cl, Br, I, alkyl, aryl, acyl, amino, carbocyclyl, heterocyclyl, heteroaryl, alkyloxy, aryloxy, acyloxy, acylamino, carbocyclyloxy, heterocyclyloxy, heteroaryloxy, alkylthio, arylthio, acylthio, carbocyclylthio, heterocyclylthio, heteroarylthio, alkylaryl, alkylacyl, alkylcarbocyclyl, alkylheterocyclyl, alkylheteroaryl, alkyloxyalkyl, aryloxyalkyl, alkylacyloxy, alkylcarbocyclyloxy, alkylheterocyclyloxy, alkylheteroaryloxy, alkylthioalkyl, arylthioalkyl, alkylacylthio, alkylcarbocyclylthio, alkylheterocyclylthio, alkylheteroarylthio, alkylarylalkyl, alkylacylalkyl, arylalkylaryl, arylacylaryl, arylacyl, arylcarbocyclyl, arylheterocyclyl; arylheteroaryl, aryloxyaryl, arylacyloxy, arylcarbocyclyloxy, arylheterocyclyloxy, arylheteroaryloxy, alkylthioaryl, arylthioaryl, arylacylthio, arylcarbocyclylthio, arylheterocyclylthio, arylheteroarylthio, dialkyloxy-, diheterocyclyloxy- or diaryloxy-phosphinyl, dialkyl-, diheterocyclyl- or diaryl-phosphinyl, cyano (i.e. —CN), and —S—R, where R is as defined in respect of formula (H).

More specific examples of Z in RAFT agents used in accordance with the invention include optionally substituted, and in the case of Z* in RAFT agents used in accordance with the invention include a y-valent form of optionally substituted: F, Cl, C₁-C₁₈ alkyl, C₆-C₁₈ aryl, C₁-C₁₈ acyl, amino, C₃-C₁₈ carbocyclyl, C₂-C₁₈ heterocyclyl, C₃-C₁₈ heteroaryl, C₁-C₁₈ alkyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₇-C₂₄ alkylaryl, C₂-C₁₈ alkyl acyl, C₄-C₁₈ alkylcarbocyclyl, C₃-C₁₈ alkylheterocyclyl, C₄-C₁₈ alkylheteroaryl, C₂-C₁₈ alkyloxyalkyl, C₇-C₂₄ aryloxyalkyl, C₂-C₁₈ alkylacyloxy, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₂₄ alkylarylalkyl, C₃-C₁₈ alkylacylalkyl, C₁₃-C₂₄ arylalkylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₇-C₁₈ alkylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, dialkyloxy-, diheterocyclyloxy- or diaryloxy-phosphinyl (i.e. —P(═O)OR^{k}₂), dialkyl-, diheterocyclyl- or diaryl-phosphinyl (i.e. —P(═O)R^{k}₂), where R^{k} is selected from optionally substituted C₁-C₁₈ alkyl, optionally substituted C₆-C₁₈ aryl, optionally substituted C₂-C₁₈ heterocyclyl, and optionally substituted C₇-C₂₄ alkylaryl, cyano (i.e. —CN), and —S—R, where R is as defined in respect of formula (II).

In one embodiment, the RAFT agent used in accordance with the invention is a trithiocarbonate RAFT agent and Z or Z* is an optionally substituted alkylthio group.

MacroRAFT agents suitable for use in accordance with the invention may obtained commercially, for example see those described in the SigmaAldrich catalogue (www.sigmaaldrich.com).

Other RAFT agents that can be used in accordance with the invention include those described in WO201083569 and Benaglia et al, Macromolecules. (42), 9384-9386, 2009, the entire contents of which are incorporated herein by reference.

In one embodiment, the at least a tri-block structure of the cationic block copolymer is formed by RAFT polymerisation. In that case; the at least a tri-block structure may be conveniently referred to as a tri-block RAFT polymer structure.

The present invention therefore also provides a complex comprising a cationic block copolymer and a nucleic acid, the cationic block copolymer having at least a tri-block RAFT polymer structure comprising a cationic block and two hydrophilic blocks, or a hydrophilic block and two cationic blocks.

In the lists herein defining groups from which Z, Z*, R and R* may be selected, each alkyl, alkenyl, alkynyl, aryl, carbocyclyl, heteroaryl, heterocyclyl, and polymer chain moiety may be optionally substituted. For avoidance of any doubt, where a given Z, Z*, R or R* contains two or more of such moieties (e.g. alkylaryl), each of such moieties may be optionally substituted with one, two, three or more optional substituents as herein defined.

In the lists herein defining groups from which Z, Z*, R and R* may be selected, where a given Z, Z*, R or R* contains two or more subgroups (e.g. [group A][group B]), the order of the subgroups is not intended to be limited to the order in which they are presented. Thus, a Z, Z*, R or R* with two subgroups defined as [group A][group B] (e.g. alkylaryl) is intended to also be a reference to a Z, Z*, R or R* with two subgroups defined as [group B][group A] (e.g. arylalkyl).

The Z, Z*, R or R* may be branched and/or optionally substituted. Where the Z, Z*, R or R* comprises an optionally substituted alkyl moiety, an optional substituent includes where a —CH₂— group in the alkyl chain is replaced by a group selected from —O—, —S—, —NR^{a}—, —C(O)— (i.e. carbonyl), —C(O)O— (i.e. ester), and —C(O)NR^{a}— (i.e. amide), where R^{a} may be selected from hydrogen, alkyl, alkenyl, alkynyl, aryl, carbocyclyl, heteroaryl, heterocyclyl, arylalkyl, and acyl.

Reference herein to a x-valent, y-valent, multi-valent or di-valent “form of . . . ” is intended to mean that the specified group is a x-valent, y-valent, multi-valent or di-valent radical, respectively. For example, where x or y is 2, the specified group is intended to be a divalent radical. In that ease, a divalent alkyl group is in effect an alkylene group (e.g. —CH₂—). Similarly, the divalent form of the group alkylaryl may, for example, be represented by —(C₆H₄)—CH₂—, a divalent alkylarylalkyl group may, for example, be represented by —CH₂—(C₆H₄)—CH₂—, a divalent alkyloxy group may, for example, be represented by —CH₂—O—, and a divalent alkyloxyalkyl group may, for example, be represented by —CH₂—O—CH₂—. Where the term “optionally substituted” is used in combination with such a x-valent, y-valent, multi-valent or di-valent groups, that group may or may not be substituted or fused as herein described. Where the x-valent, y-valent, multi-valent, di-valent groups comprise two or more subgroups, for example [group A][group B][group C] (e.g. alkylarylalkyl), if viable one or more of such subgroups may be optionally substituted. Those skilled in the art will appreciate how to apply this rationale in providing for higher valent forms.

The cationic block copolymer will generally be prepared by the polymerisation of ethylenically unsaturated monomers. Factors that determine copolymerisability of ethylenically unsaturated monomers are well documented in the art. For example, see: Greenlee, R. Z., in Polymer Handbook 3^{rd} edition (Brandup, J, and Immergut. E. H. Eds) Wiley: New York, 1989, p II/53.

Suitable examples of ethylenically unsaturated monomers that may be used to prepare the cationic block copolymer include those of formula (III):

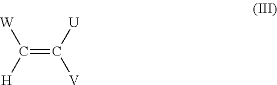

where U and W are independently selected from —CO₂H, —CO₂R¹, —COR¹, —CSR¹, —CSOR¹, —COSR¹, —CONH₂, —CONHR¹, —CONR¹₂, hydrogen, halogen and optionally substituted C₁-C₄ alkyl or U and W form together a lactone, anhydride or imide ring that may itself be optionally substituted, where the optional substituents are independently selected from hydroxy, —CO₂H, —CO₂R¹, —COR¹, —CSR¹, —CSOR¹, —COSR¹, —CN, —CONH₂, —CONHR¹, —CONR¹₂, —OR¹, —SR¹, —O₂CR¹, —SCOR¹, and —OCSR¹;
V is selected from hydrogen, R¹, —CO₂H, —CO₂R¹, —COR¹, —CSR¹, —CSOR¹, —COSR¹, —CONH₂, —CONHR¹, —CONR¹₂, —OR¹, —SR¹, —O₂CR¹, —SCOR¹, and —OCSR¹;
where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

The or each R¹ may also be independently selected from optionally substituted C₁-C₂₂ alkyl, optionally substituted C₂-C₂₂ alkenyl, optionally substituted C₂-C₂₂ alkynyl, optionally substituted C₆-C₁₈ aryl, optionally substituted C₃-C₁₈ heteroaryl, optionally substituted C₃-C₁₈ carbocyclyl, optionally substituted C₂-C₁₈ heterocyclyl, optionally substituted C₇-C₂₄ arylalkyl, optionally substituted C₄-C₁₈ heteroarylalkyl, optionally substituted C₇-C₂₄ alkylaryl, optionally substituted C₄-C₁₈ alkylheteroaryl, and an optionally substituted polymer chain.

Examples of optional substituents for R¹ include those selected from alkyleneoxidyl (epoxy), hydroxy, alkoxy, acyl, acyloxy, formyl, alkylcarbonyl, carboxy, sulfonic acid, alkoxy- or aryloxy-carbonyl, isocyanato, cyano, silyl, halo, amine (primary, secondary and tertiary), including salts and derivatives thereof.

In one embodiment R¹ is a polymer chain. Examples of polymer chains include those selected from polyalkylene oxide, polyarylene ether and polyalkylene ether.

In one embodiment, R¹ may be independently selected from amine substituted C₁-C₆ alkyl and an optionally substituted polymer chain.

Examples of monomers of formula (III) include maleic anhydride, N-alkylmaleimide, N-arylmaleimide, dialkyl fumarate and cyclopolymerisable monomers, acrylate and methacrylate esters, acrylic and methacrylic acid, styrene, styrenics, methacrylamide, and methacrylonitrile, mixtures of these monomers, and mixtures of these monomers with other monomers.

Other examples of monomers of formula (III) include: methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, oligo (ethylene glycol) methyl ether methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, N-phenylmaleimide, N-butylmaleimide, N-vinylpyrrolidone, N-vinylcarbazole, butadiene, ethylene and chloroprene. This list is not exhaustive.

When discussing the types of monomers that may be used to prepare the cationic block copolymer, it may be convenient to refer to the monomers as being hydrophilic or hydrophobic in character. For example, each of the two hydrophilic blocks of the tri-block structure will generally be prepared by polymerising a monomer composition that comprises hydrophilic monomers.

As a guide only, examples of hydrophobic ethylenically unsaturated monomers include, but are not limited to, styrene, alpha-methyl styrene, butyl acrylate, butyl methacrylate, amyl methacrylate, hexyl methacrylate, lauryl methacrylate, stearyl methacrylate, ethyl hexyl methacrylate, crotyl methacrylate, cinnamyl methacrylate, oleyl methacrylate, ricinoleyl methacrylate, cholesteryl methacrylates, cholesteryl acrylate, vinyl butyrate, vinyl tert-butyrate, vinyl stearate and vinyl laurate.

As a guide only, examples of hydrophilic ethylenically unsaturated monomers include, but are not limited to, acrylic acid, methacrylic acid, hydroxyethyl methacrylate, hydroxypropyl methacrylate, oligo(alkylene glycol)methyl ether (meth)acrylate (OAG(M)A), acrylamide and methacrylamide, hydroxyethyl acrylate, N-methylacrylamide, N,N-dimethylacrylamide and N,N-dimethylaminoethyl methacrylate, N,N-dimethylaminopropyl methacrylamide, N-hydroxypropyl methacrylamide, 4-acryloylmorpholine, 2-acrylamido-2-methyl-1-propanesulfonic acid, phosphorylcholine methacrylate and N-vinyl pyrolidone.

In the case of the hydrophilic ethylenically unsaturated monomer OAG(M)A, the alkylene moiety will generally be a C₂-C₆, for example a C₂ or C₃, alkylene moiety. Those skilled in the art will appreciate that the “oligo” nomenclature associated with the “(alkylene glycol)” refers to the presence of a plurality of alkylene glycol units. Generally, the oligo component of the OAG(M)A will comprise about 2 to about 200, for example from about 2 to about 100, or from about 2 to about 50 or from about 2 to about 20 alkylene glycol repeat units.

As a guide only, examples of ethylenically unsaturated monomers that may be used in preparing a cationic block of the cationic block copolymer include, but are not limited to, 2-aminoethyl methacrylate hydrochloride, N-[3-(N,N-dimethylamino)propyl]methacrylamide, N-(3-aminopropyl)methacrylamide hydrochloride, N-[3-(N,N-dimethylamino)propyl]acrylamide; N[2-(N,N-dimethylamino)ethyl]methacrylamide, 2-N-morpholinoethyl acrylate, 2-N-morpholinoethyl methacrylate, 2-(N,N-dimethylamino)ethyl acrylate, 2-(N,N-dimethylamino)ethyl methacrylate, 2-(N,N-diethylamino)ethyl methacrylate, 2-Acryloxyyethyltrimethylammonium chloride, methacrylamidopropyltrimethylammonium chloride, 2-(tert-butylamino)ethyl methacrylate, diallyldimethylammonium chloride, 2-(diethylamino)ethylstyrene, 2-vinylpyridine, and 4-vinylpyridine.

Where a free radical polymerisation technique is to be used in polymerising one or more ethylenically unsaturated monomers so as to form cationic block copolymers, the polymerisation will usually require initiation from a source of free radicals.

A source of initiating radicals can be provided by any suitable means of generating free radicals, such as the thermally induced homolytic scission of suitable compound(s) (thermal initiators such as peroxides, peroxyesters, or azo compounds), the spontaneous generation from monomers (e.g. styrene), redox initiating systems, photochemical initiating systems or high energy radiation such as electron beam, X- or gamma-radiation.

Thermal initiators are generally chosen to have an appropriate half life at the temperature of polymerisation. These initiators can include one or more of the following compounds:
2,2′-azobis(isobutyronitrile), 2,2′-azobis(2-cyanobutane), dimethyl 2,2′-azobis(isobutyrate), 4,4′-azobis(4-cyanovaleric acid), 1,1′-azobis(cyclohexanecarbonitrile), 2-(t-butylazo)-2-cyanopropane, 2,2′-azobis{2-methyl-N-[1,1-bis(hydroxymethyl)-2-hydroxyethyl]propionamide}, 2,2′-azobis[2-methyl-N-(2-hydroxyethyl)propionamide], 2,2′-azobis(N,N′-dimethyleneisobutyramidine) dihydrochloride, 2,2′-azobis(2-amidinopropane) dihydrochloride, 2,2′-azobis(N,N′-dimethyleneisobutyramidine), 2,2′-azobis{2-methyl-N-[1,1-bis(hydroxymethyl)-2-hydroxyethyl]propionamide}, 2,2′-azobis{2-methyl-N-[1,1-bis(hydroxymethyl)-2-ethyl]propionamide}, 2,2′-azobis[2-methyl-N-(2-hydroxyethyl)propionamide], 2,2′-azobis(isobutyramide) dihydrate, 2,2′-azobis(2,2,4-trimethylpentane), 2,2′-azobis(2-methylpropane), t-butyl peroxyacetate, t-butyl peroxybenzoate, t-butyl peroxyneodecanoate, t-butylperoxy isobutyrate, t-amyl peroxypivalate, t-butyl peroxypivalate, diisopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl hyponitrite, dicumyl hyponitrite. This list is not exhaustive.

Photochemical initiator systems are generally chosen to have an appropriate quantum yield for radical production under the conditions of the polymerisation. Examples include benzoin derivatives, benzophenone, acyl phosphine oxides, and photo-redox systems.

Redox initiator systems are generally chosen to have an appropriate rate of radical production under the conditions of the polymerisation; these initiating systems can include, but are not limited to, combinations of the following oxidants and reductants:
oxidants: potassium, peroxydisulfate, hydrogen peroxide, t-butyl hydroperoxide.
reductants: iron (II), titanium (III), potassium thiosulfite, potassium bisulfite.

Other suitable initiating systems are described in commonly available texts. See, for example, Moad and Solomon “The Chemistry of Free Radical Polymerisation”, Pergamon, London, 1995, pp 53-95.

Initiators that are more readily solvated in hydrophilic media include, but are not limited to, 4,4-azobis(cyanovaleric acid), 2,2′-azobis{2-methyl-N-[1,1-bis(hydroxymethyl), 2-hydroxyethyl]propionamide}, 2,2′-azobis[2-methyl-N-(2-hydroxyethyl)propionamide], 2,2′-azobis(N,N′-dimethyleneisobutyramidine), 2,2′-azobis(N,N′-dimethyleneisobutyramidine)dihydrochloride, 2,2′-azobis(2-amidinopropane) dihydrochloride, 2,2′-azobis{2-methyl-N-[1,1-bis(hydroxymethyl)-2-ethyl]propionamide}, 2,2′-azobis[2-methyl-N-(2-hydroxyethyl)propionamide], 2,2′-azobis(isobutyramide) dihydrate, and derivatives thereof.

Initiators that are more readily solvated in hydrophobic media include azo compounds exemplified by the well known material 2,2′-azobisisobutyronitrile. Other suitable initiator compounds include the acyl peroxide class such as acetyl and benzoyl peroxide as well as alkyl peroxides such as cumyl and t-butyl peroxides. Hydroperoxides such as t-butyl and cumyl hydroperoxides are also widely used.

In one embodiment, the cationic block copolymer is prepared by free radical polymerisation using a bis-trithiocarbonate RAFT agent. In that case, the RAFT agent is used to first polymerise a monomer composition comprising monomer that will provide for the cationic block. For example, the monomer composition may comprise an amine substituted (meth)acrylate such as N,N-dimethyl amino alkyl (meth)acrylate. The polymerisation provides for a telechelic macroRAFT agent comprising the block that will subsequently be converted in to the cationic block. A second polymerisation step is then conducted whereby the telechelic macroRAFT agent is used to polymerise a monomer composition comprising hydrophilic monomer so as to provide for each of the two hydrophilic blocks. For example, the monomer composition may comprise oligo(alkylene glycol) methyl ether (meth)acrylate such as oligo (ethylene glycol) methyl ether (meth)acrylate. The monomer composition polymerised to form each of the two hydrophilic blocks may also comprise a mixture of two or more different monomers so as to provide for a copolymer hydrophilic block. For example, the monomer composition polymerised to form each of the two hydrophilic blocks may comprise a mixture of oligo (ethylene glycol) methyl ether (meth)acrylate and N,N-dimethyl amino ethyl (meth)acrylate.

According to this embodiment, the resulting polymer has an A-B-A tri-block structure. In its polymerised form, the A block comprises monomer residue units having tertiary amino groups that are subsequently quaternarised in a further step so as to afford the charged cationic block of the cationic block copolymer. The resulting cationic block copolymer will have a structure as shown below in general formula (IV):

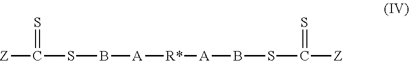

where Z, B, A and R* are as herein defined.

The present invention also provides a method of delivering a nucleic acid to a cell, the method comprising preparing a complex comprising a cationic block copolymer and a nucleic acid, the cationic block copolymer having at least a tri-block structure comprising a cationic block and two hydrophilic blocks, and introducing the complex to the cell. The method may be performed in vivo, ex vivo or in vitro.

The present invention further provides a method of gene therapy comprising the administration to a subject in need thereof a therapeutically effective amount of the nucleic acid complex according to the present invention, as herein described.

The relevance of DNA repair and mediated recombination as gene therapy is apparent when studied, for example, in the context of genetic diseases such as cystic fibrosis, hemophilia and globinopathies such as sickle cell anemia and beta-thalassemia. For example, if the target gene contains a mutation that is the cause of a genetic disorder, then introducing a nucleic acid into the cell(s) of a subject can be useful for facilitating mutagenic repair to restore the DNA sequence of the abnormal target gene to normal. Alternatively, the nucleic acid introduced to the cell(s) of a subject may lead to the expression of a gene that is otherwise suppressed or silent in the disease state. Such nucleic acids may themselves encode the silent or suppressed gene, or they may activate transcription and/or translation of an otherwise suppressed or silent target gene.

It would be understood by those skilled in the art that the disease or condition to be treated using the method of the present invention may be any disease or condition capable of treatment by gene therapy and the choice of the genetic material (i.e., nucleic acid) to be used will clearly depend upon the particular disease or condition. Diseases or conditions that may be treated include, but are not limited to, cancers (e.g. myeloid disorders), thalassemia, cystic fibrosis, deafness, vision disorders (e.g. Leber's congenital amaurosis), diabetes, Huntingdon's disease, X-linked severe combined immunodeficiency disease and heart disease. Alternatively, the gene therapy may be used to introduce non-endogenous genes, for example, genes for bioluminescence, or to introduce genes which will knock out endogenous genes (e.g. RNA interference).

It would also be understood by those skilled in the art that the nature of the nucleic acid will invariably depend on the disease or condition to be treated or prevented. For example, a disease or condition that is attributed, at least in part, to an accumulation of fibrotic extracellular matrix material (e.g., type II collagen), can be treated or prevented by delivering the nucleic acid complex of the present invention to the subject (in a targeted or non-targeted approach), wherein the nucleic acid molecule (e.g., siRNA) is capable of silencing the gene that encodes the extracellular matrix material. In some embodiments, the disease or condition is an infectious disease, an inflammatory disease, or a cancer.

Where delivery of the nucleic acid complex to a cell in accordance with the present invention is performed in vivo, the nucleic acid complex can be introduced to the cell by any route of administration that is appropriate under the circumstances. For instance, where systemic delivery is intended, the complex may be administered intravenously, subcutaneously, intramuscularly, orally, etc. Alternatively, the complex may be targeted to a particular cell or cell type by means known to those skilled in the art. Targeting may be desirable for a variety of reasons such as, for example, to target cancer cells if the nucleic acid molecule is unacceptably toxic to non-cancerous cells or if it would otherwise require too high a dosage. Targeted delivery may be achieved by any means know to those skilled in the art including, but not limited to, receptor-mediated targeting or by administering the nucleic acid complex directly to the tissue comprising the target cell(s).

Receptor-mediated targeting may be achieved, for example, by conjugating the nucleic acid molecule to a protein ligand, e.g., via polylysine. Ligands are typically chosen on the basis of the presence of the corresponding ligand receptors on the surface of the target cell/tissue type. These ligand-nucleic acid conjugates can be complexed with a cationic block copolymer in accordance with the present invention and administered systemically if desired (e.g., intravenously), where they will be directed to the target cell/tissue where receptor binding occurs.

In one embodiment, the method of delivering a nucleic acid to a cell in accordance with the present invention is performed ex vivo. For example, cells are isolated from the subject and introduced ex vivo with the nucleic acid complex of the present invention to produce cells comprising the exogenous nucleic acid. The cells may be isolated from the subject to be treated or from a syngeneic host. The cells are then reintroduced back into the subject (or into a syngeneic recipient) for the purpose of treatment or prophyaxis. In some embodiments, the cells can be hematopoietic progenitor or stem cells.

In one embodiment, the nucleic acid is delivered to a cell for the purpose of silencing (or suppressing) gene expression. In some embodiments, gene expression is silenced by reducing translational efficiency or reducing message stability or a combination of these effects. In some embodiments, splicing of the unprocessed RNA is the target goal leading to the production of non-functional or less active protein.

For example, the method of the invention may be used for reducing viral replication. In such an embodiment the nucleic acid will be capable of (or is selected for) silencing the expression of a virus derived gene in the cell.

In some embodiments, gene expression is silenced by introducing to a cell a DNA molecule, including but not limited to, gDNA, cDNA and DNA oligonucleotides (double or single stranded).

In some embodiments, gene expression is silenced by RNA interference (RNAi). Without limiting the present invention to a particular theory or mode of action, “RNA interference” typically describes a mechanism of silencing gene expression that is based on degrading or otherwise preventing the translation of mRNA, for example, in a sequence specific manner. It would be understood by those skilled in the art that the exogenous interfering RNA molecules may lead to either mRNA degradation or mRNA translation repression. In some embodiments, RNA interference is achieved by altering the reading frame to introduce one or more premature stop codons that lead to non-sense mediated decay.

RNAi includes the process of gene silencing involving double stranded (sense and antisense) RNA that leads to sequence specific reduction in gene expression via target mRNA degradation. RNAi is typically mediated by short double stranded siRNAs or single stranded microRNAs (miRNA). In some embodiments, RNAi is initiated when a strand of RNA from either or these molecules forms a complex referred to as an RNA-induced silencing complex (RISC) which targets complementary RNA and suppresses translation. The process can be exploited for research purposes and for therapeutic application (see for example, Izquierdo et al., Cancer Gene Therapy, 12(3): 217-27, 2005).

Other oligonucleotides having RNA-like properties have also been described and many more different types of RNAi may be developed. For example, antisense oligonucleotides have been used to alter exon usage and to modulate pre-RNA splicing (see, for example, Madocsai et al., Molecular Therapy, 12: 1013-1022, 2005 and Aartsma-Rus et al., BMC Med Genet., 8: 43, 2007). Antisense and iRNA compounds may be double stranded or single stranded oligonucleotides which are RNA or RNA-like or DNA or DNA-like molecules that hybridize specifically to DNA or RNA of the target gene of interest.

Examples of RNA molecules suitable for use in the context of the present invention include, but are not limited to:
(i) long double stranded RNA (dsRNA)—these are generally produced as a result of the hybridisation of a sense RNA strand and an antisense RNA strand which are each separately transcribed by their own vector. Such double stranded molecules are typically not characterised by a hairpin loop. These molecules are required to be cleaved by an enzyme such as Dicer in order to generate short interfering RNA (siRNA) duplexes. This cleavage event preferably occurs in the cell in which the dsRNA is transcribed.
(ii) hairpin double stranded RNA (hairpin dsRNA)—these molecules exhibit a stem-loop configuration and are generally the result of the transcription of a construct with inverted repeat sequences which are separated by a nucleotide spacer region, such as an intron. These molecules are generally of longer RNA molecules which require both the hairpin loop to be cleaved off and the resultant linear double stranded molecules to be cleaved by the enzyme Dicer in order to generate siRNA. This type of molecule has the advantage of being expressible by a single vector.
(iii) short interfering RNA (siRNA)—these can be synthetically generated or, recombinantly expressed by the promoter based expression of a vector comprising tandem sense and antisense strands each characterised by its own promoter and a 4-5 thymidine transcription termination site. This enables the generation of two separate transcripts which subsequently anneal. In some embodiments, these transcripts may be of the order of 20-25 nucleotides in length. *Accordingly, these molecules require no further cleavage to enable their functionality in the RNA interference pathway.
(iv) short hairpin RNA (shRNA)—these molecules are also known as “small hairpin RNA” and are typically similar in length to the siRNA molecules but with the exception that they comprise inverted repeat sequences of an RNA molecule, the inverted repeats being separated by a nucleotide spacer. Subsequently to the cleavage of the hairpin (loop) region, a functional siRNA molecule is generated.
(v) micro RNA/small temporal RNA (miRNA/stRNA)—miRNA and stRNA are generally understood to represent naturally-occurring, endogenously expressed molecules. Accordingly, although the design and administration of a molecule intended to mimic the activity of a miRNA will take the form of a synthetically generated or recombinantly expressed siRNA molecule, the present invention nevertheless extends to the design and expression of oligonucleotides intended to mimic miRNA, pri-miRNA or pre-miRNA molecules by virtue of exhibiting essentially identical RNA sequences and overall structure. Such recombinantly generated molecules may be referred to as either miRNAs or siRNAs.
(vi) miRNAs which mediate spatial development (sdRNAs), the stress response (srRNAs) or cell cycle (ccRNAs).
(vii) RNA oligonucleotides designed to hybridise and prevent the functioning of endogenously expressed miRNA or stRNA or exogenously introduced siRNA. In some embodiments, it would be appreciated that these molecules are not designed to invoke the RNA interference mechanism but, rather, prevent the upregulation of this pathway by the miRNA and/or siRNA molecules which are present in the intracellular environment. In terms of their effect on the miRNA to which they hybridise, this is reflective of more classical antisense inhibition.

Reference to an “RNA oligonucleotide” should be understood as a reference to an RNA nucleic acid molecule which is double stranded or single stranded and is capable of either inducing an RNA interference mechanism directed to silencing the expression of a target gene. In this regard, the subject oligonucleotide may be capable of directly modulating an RNA interference mechanism or it may require further processing, such as is characteristic of (i) hairpin double stranded RNA, which requires excision of the hairpin region, (ii) longer double stranded RNA molecules which require cleavage by dicer or (iii) precursor molecules such as pre-miRNA, which similarly require cleavage. The subject oligonucleotide may be double stranded (as is typical in the context of effecting RNA interference) or single stranded (as may be the case if one is seeking only to produce a RNA oligonucleotide suitable for binding to an endogenously expressed gene).

In other embodiments, the nucleic acid molecule suppresses translation initiation, splicing at a splice donor site or splice acceptor site. In other embodiments, modification of splicing alters the reading frame and initiates nonsense mediated degradation of the transcript.

It will be appreciated that a person of skill in the art can determine the most suitable nucleic acid molecule for use in accordance with the present invention and for any given situation. For example, although it is preferable that an RNA molecule exhibits 100% complementarity to its target nucleic acid sequence, the RNA molecule may exhibit some degree of mismatch to the extent that hybridisation sufficient to induce an RNA interference response in a sequence-specific manner is enabled. Accordingly, it is preferred that the RNA molecule comprises at least 70% sequence complementarity, more preferably at least 90% complementarity and even more preferably, 95%, 96%, 97%, 98% 99% or 100% sequence complementarity with the target nucleic acid sequence.

In another example pertaining to the design of a nucleic acid molecule suitable for use in accordance with the present invention, it is within the skill of the person of skill in the art to determine the particular structure and length of the molecule, for example whether it takes the form of dsRNA, hairpin dsRNA, siRNA, shRNA, miRNA, pre-miRNA, pri-miRNA or any other suitable form as herein described. For example, it is generally understood that stem-loop RNA structures, such as hairpin dsRNA and shRNA, are typically more efficient in terms of achieving gene silencing than, for example, double stranded DNA which is generated utilising two constructs separately coding the sense and antisense RNA strands. Furthermore, the nature and length of the intervening spacer region can impact on the functionality of a given stem-loop RNA molecule. In yet another example, the choice of long dsRNA, which requires cleavage by an enzyme such as Dicer, or short dsRNA (such as siRNA or shRNA) can be relevant if there is a risk that in the context of the particular cellular environment, an interferon response could be generated, this being a more significant risk where long dsRNA is used than where short dsRNA molecules are utilised. In still yet another example, whether a single stranded or double stranded nucleic acid molecule is required to be used will also depend on the functional outcome which is sought. For example, to the extent that one is targeting an endogenously expressed miRNA with an antisense molecule, it would generally be appropriate to design a single stranded RNA oligonucleotide suitable for specifically hybridising to the subject miRNA. To the extent that it is sought to induce RNA interference, a double stranded siRNA molecule may be required. In some embodiments, this may be designed as a long dsRNA molecule which undergoes further cleavage or an siRNA.

The term “gene” is used in its broadest sense and includes cDNA corresponding to the exons of a gene. Reference herein to a “gene” is also taken to include: a classical genomic gene consisting of transcriptional and/or translational regulatory sequences and/or a coding region and/or non-translated sequences (i.e. introns, 5′- and 3′-untranslated sequences); or an mRNA or cDNA molecule corresponding to the coding regions (i.e. exons), pre-mRNA and 5′- and 3′-untranslated sequences of the gene.

Reference to “expression” is a broad reference to gene expression and includes any stage in the process of producing protein or RNA from a gene or nucleic acid molecule, from pre-transcription, through transcription and translation to post-translation.

A “cell”, as used herein, includes a eukaryotic cell (e.g., animal cell, plant cell and a cell of fungi or protists) and a prokaryotic cell (e.g., a bacterium). In one embodiment, the cell is a human cell.

The term “subject”, as used herein, means either an animal or human subject. By “animal” is meant primates, livestock animals (including cows, horses, sheep, pigs and goats), companion animals (including dogs, cats, rabbits and guinea pigs), captive wild animals (including those commonly found in a zoo environment), and aquatic animals (including freshwater and saltwater animals such as fish and crustaceans. Laboratory animals such as rabbits, mice, rats, guinea pigs and hamsters are also contemplated as they may provide a convenient test system. In some embodiments, the subject is a human subject.

By “administration” of the complex or composition to a subject is meant that the agent or composition is presented such that it can be or is transferred to the subject. There is no particular limitation on the mode of administration, but this will generally be by way of oral, parenteral (including subcutaneous, intradermal, intramuscular, intravenous, intrathecal, and intraspinal), inhalation (including nebulisation), rectal and vaginal modes.

Without being bound or limited by theory, the complex of the present invention has been found to protect the nucleic acid molecule from degradation by enzymes such as RNAse and/or DNAse. The present invention therefore also provides a method of protecting a nucleic acid form enzymatic degradation, the method comprising complexing the nucleic acid with a cationic block copolymer, the cationic block copolymer having at least a tri-block structure comprising a cationic block and two hydrophilic blocks, or a hydrophilic block and two cationic blocks.

There is also provided use of a complex for delivering a nucleic acid to a cell, the complex comprising a cationic block copolymer and the nucleic acid, the cationic block copolymer having at least a tri-block structure comprising a cationic block and two hydrophilic blocks, or a hydrophilic block and two cationic blocks.

The present invention further provides use of a complex for silencing gene expression, the complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

In one embodiment, the DNA and RNA are selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear (SnRNAs) ribozymes, aptamers, DNAzymes, ribonuclease-type complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

Without being bound or limited by theory, the complex of the present invention has been found to protect the nucleic acid molecule from degradation by enzymes such as RNAse and/or DNAse. The present invention therefore provides use of a cationic block copolymer in protecting a nucleic acid from enzymatic degradation, the cationic block copolymer having at least a tri-block structure comprising a cationic block and two hydrophilic blocks, or a hydrophilic block and two cationic blocks.

The complex in accordance with the invention may also be used in the manufacture of compositions, such as pharmaceutical compositions, for delivering a nucleic acid to a cell and/or for silencing gene expression.

The invention therefore also provides use of a complex in the manufacture of a composition for delivering a nucleic acid to a cell, the complex comprising a cationic block copolymer and the nucleic acid, the cationic block copolymer having at least a tri-block structure comprising a cationic block and two hydrophilic blocks, or a hydrophilic block and two cationic blocks.

The invention further provides use of a complex in the manufacture of a composition for silencing gene expression, the complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

The cationic block copolymer may also be used in protecting a nucleic acid from enzymatic degradation, the cationic block copolymer having at least a tri-block structure comprising a cationic block and two hydrophilic blocks, or a hydrophilic block and two cationic blocks. In such an embodiment, the cationic block copolymer may be seen to function as a stabilising agent.

The present invention is also directed to compositions, such as pharmaceutical compositions, comprising the nucleic acid complex of the present invention. In some embodiments, the composition will comprise the nucleic acid complex of the present invention and one or more pharmaceutically acceptable carriers, diluents and/or excipients.

In the compositions of the present invention, the nucleic acid complex is typically formulated for administration in an effective amount. The terms “effective amount” and “therapeutically effective amount” of the nucleic acid complex as used herein typically mean a sufficient amount of the complex to provide in the course the desired therapeutic or prophylactic effect in at least a statistically significant number of subjects. Undesirable effects, e.g. side effects, are sometimes manifested along with the desired therapeutic effect; hence, a practitioner would typically balance the potential benefits against the potential risks in determining what is an appropriate “effective amount”. The exact amount required will also vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

In some embodiments, an effective amount for a human subject lies in the range of about 0.1 ng/kg body weight/dose to 1 g/kg body weight/dose. In some embodiments, the range is about 1 μg to 1 g, about 1 mg to 1 g, 1 mg to 500 mg, 1 mg to 250 mg, 1 mg to 50 mg, or 1 μg to 1 mg/kg body weight/dose. Dosage regimes are adjusted to suit the exigencies of the situation and may be adjusted to produce the optimum therapeutic or prophylactic dose. For example, several doses may be provided daily, weekly, monthly or other appropriate time intervals. Thus, the time and conditions sufficient for transfection can be determined by one skilled such as a medical practitioner who is able to specify a therapeutically or prophylactively effective amount.

By “pharmaceutically acceptable” carrier, excipient or diluent is meant a pharmaceutical vehicle comprised of a material that is not biologically or otherwise undesirable; that is, the material may be administered to a subject along with the complex of the present invention without causing any or a substantial adverse reaction. Carriers may include excipients and other additives such as diluents, detergents, colouring agents, wetting or emulsifying agents, pH buffering agents, preservatives, and the like.

Aspects of the present invention include methods for treating a subject for an infectious disease, an inflammatory disease, or a cancer, the method comprising administering to the subject a complex according to the invention, or a pharmaceutical composition according to the invention, to the subject.

The a cationic block copolymer according to the present invention has advantageously been found to not only function as a transfection agent, but also as a delivery agent and as a stabilising agent.

As used herein, the term “alkyl”, used either alone or in compound words denotes straight chain, branched or cyclic alkyl, preferably C₁₋₂₀ alkyl, e.g. C₁₋₁₀ or C₁₋₆. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or polyunsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined. Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substituents as herein defined.

The term “halogen” (“halo”) denotes fluorine, chlorine, bromine or iodine (fluoro, chloro, bromo or iodo).

The term “aryl” (or “carboaryl”) denotes any of single, polynuclear, conjugated and fused residues of aromatic hydrocarbon ring systems(e.g. C₆₋₂₄ or C₆₋₁₈). Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may or may not be optionally substituted by one or more optional substituents as herein defined. The term “arylene” is intended to denote the divalent form of aryl.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl. A carbocyclyl group may be optionally substituted by one or more optional substituents as herein defined. The term “carbocyclylene” is intended to denote the divalent form of carbocyclyl.

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

The term “heterocyclyl” when used alone or in compound words includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include o, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds.

Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl. A heterocyclyl group may be optionally substituted by one or more optional substituents as herein defined. The term “heterocyclylene” is intended to denote the divalent form of heterocyclyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl. A heteroaryl group may be optionally substituted by one or more optional substituents as herein defined. The term “heteroarylene” is intended to denote the divalent form of heteroaryl.

The term “acyl” either alone or in compound words denotes a group containing the moiety C═O (and not being a carboxylic acid, ester or amide) Preferred acyl includes C(O)—R^{e}, wherein R^{e} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R^{e} residue may be optionally substituted as described herein.

The term “sulfoxide”, either alone or in a compound word, refers to a group —S(O)R^{f} wherein R^{f} is selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl.

The term “sulfonyl”, either alone or in a compound word, refers to a group S(O)₂—R^{f}, wherein R^{f} is selected from hydrogen, alkyl, alkenyl, alkynyl, aryl, heteroaryl, heterocyclyl, carbocyclyl and aralkyl. Examples of preferred R^{f} include C₁₋₂₀alkyl, phenyl and benzyl.

The term “sulfonamide”, either alone or in a compound word, refers to a group S(O)NR^{f}R^{f} wherein each R^{f} is independently selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl. In one embodiment at least one R^{f} is hydrogen. In another embodiment, both R^{f} are hydrogen.

The term, “amino” is used here in its broadest sense as understood in the art and includes groups of the formula NR^{a}R^{b} wherein R^{a} and R^{b} may be any independently selected from hydrogen, alkyl, alkenyl, alkynyl, aryl, carbocyclyl, heteroaryl, heterocyclyl, arylalkyl, and acyl. R^{a} and R^{b}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋₂₀alkyl), NHaryl (e.g. NHphenyl), NHaralkyl (e.g. NHbenzyl), NHacyl (e.g. NHC(O)C₁₋₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{a}R^{b}, wherein R^{a} and R^{b} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g. C(O)NHC(O)C₁₋₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term “carboxy ester” is used here in its broadest sense as understood in the art and includes groups having the formula CO₂R^{g}, wherein R^{g} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g. CO₂-phenyl), CO₂aralkyl (e.g. CO₂ benzyl).

As used herein, the term “aryloxy” refers to an “aryl” group attached through an oxygen bridge. Examples of aryloxy substituents include phenoxy, biphenyloxy, naphthyloxy and the like.

As used herein, the term “acyloxy” refers to an “acyl” group wherein the “acyl” group is in turn attached through an oxygen atom. Examples of “acyloxy” include hexylcarbonyloxy (heptanoyloxy), cyclopentylcarbonyloxy, benzoyloxy, 4-chlorobenzoyloxy, decylcarbonyloxy (undecanoyloxy), propylcarbonyloxy (butanoyloxy), octylcarbonyloxy (nonanoyloxy), biphenylcarbonyloxy (eg 4-phenylbenzoyloxy), naphthylcarbonyloxy (eg 1-naphthoyloxy) and the like.

As used herein, the term “alkyloxycarbonyl” refers to a “alkyloxy” group attached through a carbonyl group. Examples of “alkyloxycarbonyl” groups include butylformate, sec-butylformate, hexylformate, octylformate, decylformate, cyclopentylformate and the like. As used herein, the term “arylalkyl” refers to groups formed from straight or branched chain alkanes substituted with an aromatic ring. Examples of arylalkyl include phenylmethyl (benzyl), phenylethyl and phenylpropyl.

As used herein, the term “alkylaryl” refers to groups formed from aryl groups substituted with a straight chain or branched alkane. Examples of alkylaryl include methylphenyl and isopropylphenyl.

In this specification “optionally substituted” is taken to mean that a group may or may not be substituted or fused (so as to form a condensed polycyclic group) with one, two, three or more of organic and inorganic groups, including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroary loxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate, phosphate, triarylmethyl, triarylamino, oxadiazole, and carbazole groups. Optional substitution may also be taken to refer to where a —CH₂— group in a chain or ring is replaced by a group selected from —O—, —S—, —NR^{a}—, —C(O)— (i.e. carbonyl), —C(O)O— (i.e. ester), and —C(O)NR^{a}— (i.e. amide), where R^{a} is as defined herein.

Preferred optional substituents include alkyl, (e.g. C₁₋₆ alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro OC(O)C₁₋₆ alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkylamino (e.g. C₁₋₆ alkyl, such as methylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), nitro, formyl, —C(O)-alkyl (e.g. C₁₋₆ alkyl, such as acetyl), O—C(O)-alkyl (e.g. C₁₋₆alkyl, such as acetyloxy), benzoyl (wherein the phenyl group itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C═O, CO₂H, CO₂alkyl (e.g. C₁₋₆ alkyl such as methyl ester, ethyl ester, propyl ester, butyl ester), CO₂-phenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl (e.g. C₁₋₆ alkyl such as methyl ester, ethyl ester, propyl ester, butyl amide) CONHdialkyl (e.g. C₁₋₆ alkyl) aminoalkyl (e.g., HN C₁₋₆ alkyl-, C₁₋₆alkylHN—C₁₋₆ alkyl- and (C₁₋₆ alkyl)₂N—C₁₋₆ alkyl-), thioalkyl (e.g., HS C₁₋₆ alkyl-), carboxyalkyl (e.g., HO₂CC₁₋₆ alkyl-), carboxyesteralkyl (e.g., C₁₋₆ alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆ alkyl(P)CC₁₋₆alkyl-), nitroalkyl (e.g., O₂NC₁₋₆ alkyl-), sulfoxidealkyl (e.g., R(O)SC₁₋₆ alkyl, such as C₁₋₆ alkyl(0)SC₁₋₆ alkyl-), sulfonylalkyl (e.g., R(O)₂SC₁₋₆ alkyl—such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-), triarylmethyl, triarylamino, oxadiazole, and carbazole.

The invention will now be described with reference to the following non-limiting examples.

### EXAMPLES

### Example 1

### Materials

N,N-Dimethylaminoethyl methacrylate (DMAEMA) and oligo(ethylene glycol) methyl ether methacrylate (OEGMA₄₇₅, Mn˜475 g mol⁻¹) monomers were purchased from Aldrich and purified by stirring in the presence of inhibitor-remover for hydroquinone or hydroquinone monomethyl ether (Aldrich) for 30 min prior to use. Bis-RAFT Agent, 4-cyano-4-(dodecylthiocarbonothioylthio)pentanoyloxy)butyl 4-cyano-4-(dodecylthiocarbonothioylthio)pentanoate (I) was prepared according to the procedure described below. 1,1′-Azobis(cyclohexanecarbonitrile) (VAZO-88) initiator (DuPont) was used as received. N,N-Dimethylformamide (DMF) (AR grade, Merck) was degassed by sparging nitrogen for at least 15 min prior to use. Dicholormethane (DCM), n-heptane, diisopropyl ether, methyl iodide and methanol and other chemicals were commercial reagents and used without further purification.

### Method

Bis-RAFT Agent: 4-cyano-4(dodecylthiocarbonothioylthio)pentanoyloxy)butyl 4-cyano-4-(dodecylthiocarbonothioylthio)pentanoate: C₄₂H₇₂N₂O₄S₆; MW 861.42

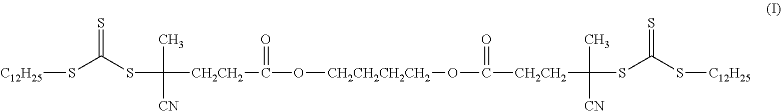

(S)-4-cyano-4-(dodecylthiocarbonothioylthio)pentanoic acid (8.1 g, 20.1 mmol), 1,4-butanediol (0.9 g, 10 mmol), DCC (dicyclohexylcarbodiimide, 4.95 g, 24.0 mmol) in dichloromethane (60 mL) and DMAP(N,N-dimethylaminopyridine, catalytic amount) were allowed to stir at room temperature for one hour. After removal of solvent, the crude reaction mixture was purified by column chromatography on a silica column using ethyl acetate:n-hexane 2:5 (v/v) as eluent to give the title product (6.2 g, 72% yield) as a yellow oil, which solidified in a refrigerator. Proton nuclear magnetic resonance (¹H NMR) (CDCl₃) (ppm) 0.89 (t, 6H, 2×CH₃); 1.27 (br s, 36H); 1.72 (m, 4H); 1.90 (s, 6H, 2×CH₃); 2.40-2.80 (m, 8H, 2×CH₂CH₂); 3.38 (t, 4H, 2×CH₂S); 4.15 (t, 4H, 2×CH₂O).

### Step 1: Synthesis and characterization of poly(N,N-dimethylaminoethyl methacrylate) (PDMAEMA) telechelic macroRAFT agent

In a typical polymerization experiment, 786 mg of DMAEMA monomer (5.00×10⁻³ mol), 0.66 mg of VAZO-88 initiator (2.70×10⁻⁶ mol), and 53.84 mg of bis-RAFT agent (6.25×10⁻⁵ mol) and 621 mg of DMF (8.49×10⁻³ mol) were mixed together in a 13 mL glass reactor of an automated parallel synthesizer (Chemspeed Swing-SLT) as follows. Stock solutions of VAZO-88 (initiator) dissolved in DMAEMA (monomer) and bis-RAFT agent dissolved in DMF were prepared and degassed by sparging nitrogen for at least 15 min prior to use. These stock solutions were added and combined into one of the reactors of the parallel synthesizer using its automated liquid handling system in order to reach the aforementioned amounts of reagents. Once in the reactor, the reaction mixture was subjected to three freeze-pump-thaw cycles between −90° C. and −10° C. (10 mbar vacuum for 2 min each cycle) in the parallel synthesizer.

Thereafter, the reaction mixture was heated up to 90° C. for 12.5 h. The monomer to polymer conversion achieved was 92% as determined by proton nuclear magnetic resonance (¹H-NMR) (in deuterated chloroform (CDCl₃)) by comparing the integration of resonance peaks in the δ4.2-4.3 ppm region, corresponding to the —CH₂ protons of the DMAEMA monomer, with that of the peaks in the δ3.9-4.2 ppm region, pertaining to the —CH₂ protons of the repeat units of the PDMAEMA polymer. The conversion was then calculated using the following equation: % Monomer conversion=[I_{3.9}/(I_{4.3}+I_{3.9})]×100; where I_{4.3} and I_{3.9} are the integral values for the —CH₂ protons of the DMAEMA monomer and of the PDMAEMA polymer, respectively. The number average molecular weight (Mn) of the polymer was 18637 Da (polydisperisty index (PDI) of 1.17) as determined by gel permeation chromatography (GPC) against polystyrene standards.

### Step 2: Synthesis and characterization of Poly(oligo(ethylene glycol) methyl ether methacrylate-block-N,N-Dimethylaminoethyl methacrylate-block-oligo(ethylene glycol) methyl ether methacrylate) (P(OEGMA₄₇₅-b-DMAEMA-b-OEGMA₄₇₅)

Two methods were used to prepare the ABA triblock copolymer. In the first method (Method A) the product at the end of Step 1 was directly used to copolymerize with OEGMA₄₇₅. In this method, the unreacted DMAEMA present is randomly copolymerized with OEGMA₄₇₅ producing a quasi-triblock copolymer. In the second method (Method B), the unreacted DMAEMA was completely removed before the second monomer is copolymerized. In this case the triblock copolymer produced has pure homopolymer blocks of DMAEAM and OEGMA₄₇₅, respectively

### Method A

The polymer solution from Step 1 (poly(N,N-dimethylaminoethyl methacrylate) (PDMAEMA) homopolymer (telechelic macroRAFT agent)) was mixed with 950 mg of OEGMA₄₇₅ (2.00×10⁻³ mol) and 0.66 mg of VAZO-88 initiator (2.70×10⁻⁶ mol) in a 13 mL glass reactor of an automated parallel synthesizer (Chemspeed Swing-SLT) as follows. Stock solutions of VAZO-88 (initiator) dissolved in OEGMA₄₇₅ (monomer) and telechelic macroRAFT agent dissolved in DMF (solvent) were prepared and degassed by sparging nitrogen for at least 15 min prior to use. These stock solutions were added and combined into one of the reactors of the parallel synthesizer using its automated liquid handling system in order to reach the aforementioned amounts of reagents. Once in the reactor, the reaction mixture was subjected to three freeze-pump-thaw cycles between −90° C. and −10° C. (10 mbar vacuum for 2 min each cycle) in the parallel synthesizer. The materials obtained from this approach are expected to have a macromolecular architecture known as quasi-triblock copolymer (since residual monomer after Step 1 was not removed). The polymer 422-3 (see Table 1) was prepared according to this method.

### Method B

The obtained reaction mixture from polymerization Step 1 was diluted with DCM and the polymer was precipitated by adding drop-wise the mixture into n-heptane; the precipitated polymer was decanted from the rest of the solution. This later procedure was carried out two times. In a final step the polymer was dried under vacuum at 40° C. until constant weight. The dried polymer (PDMAEMA homopolymer (telechelic macroRAFT agent)) was redissolved in 2638 mg of DMF (3.61×10⁻² mol). VAZO-88 (initiator) dissolved in OEGMA₄₇₅ (monomer) were added into this later solution and exposed to similar conditions as above mentioned for the case of the synthesis of quasi-triblock copolymers. The material obtained from this approach is expected to have a macromolecular architecture known as triblock copolymer (since residual monomer of polymerization in step 1 was removed by the explained precipitation procedure). Polymer 1125 (see Table 1) was prepared using this method.

The reaction mixtures in all cases in Step 2 (Method A and B) were heated up to 90° C. for 6 h. The monomer to polymer conversion achieved was 78% as determined by ¹H-NMR (in CDCl₃; following a similar procedure as explained above for the polymerization of DMAEMA).

### Purification of Triblock Copolymers:

The polymerized reaction mixture after Step 2 (Method A or B) was diluted with DCM and the polymer was precipitated by adding drop-wise the mixture into diisopropyl ether; the precipitated polymer was decanted from the rest of the solution. This later procedure was carried out two times. In a final step the polymer was dried under vacuum until constant weight. Further purification of the polymeric material was carried out by dialysis (molecular weight cut-off of 3500, Spectra Por, Spectrum Medical Industries, Inc., Houston, Tx) against MilliQ water for 3 days. After dialysis, the water was removed from the aqueous polymer solution in a Rotovapor Evaporator.

### Quaternisation:

The polymer was redissolved in DCM and an excess of methyl iodide was added into this solution and stirred for 2 h at room temperature in order to achieve the quaternization of the tertiary amino groups of PDMAEMA block of the triblock copolymer. In a final step, DCM and excess of methyl iodide were removed in a Rotovapor Evaporator; the polymer was further dried under vacuum at 40° C. for 24 h.

By using a similar procedure but with different monomer feed ratios, several triblock copolymers with different lengths of both cationic (PDMAEMA) and hydrophilic (P(OEGMA₄₇₅)) blocks were prepared. Polymer samples 422-1 422-2, 1007-1, 1007-2 and 1007-3 were prepared using Method A in Step 2 (see Table 1).

The general chemical structure (A) of the triblock copolymers is shown below, whereas Table 1 summarizes the molecular weight and block lengths of the series of block copolymers prepared.

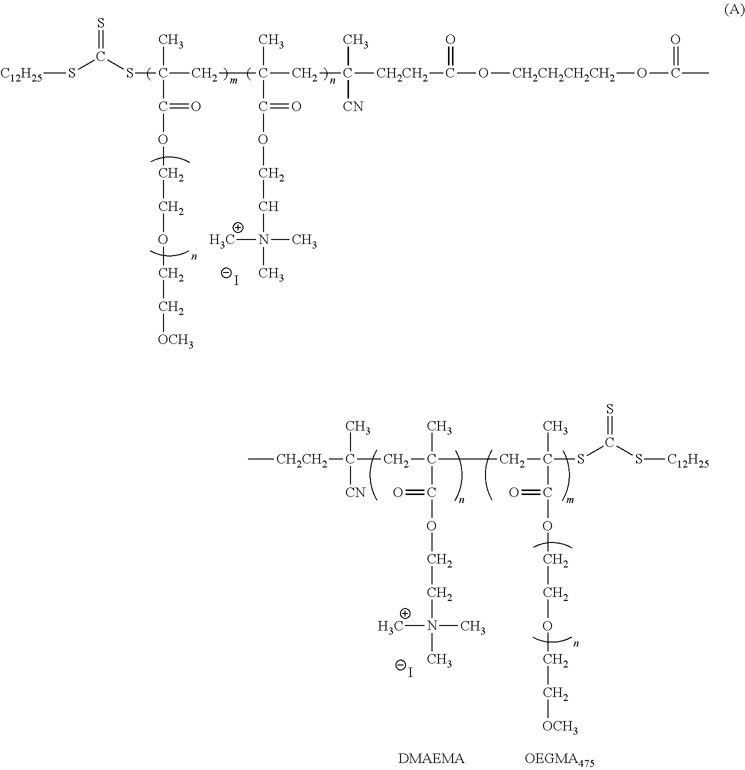

TABLE 1
ABA block copolymers prepared in Example 1

| | Experimental | | # of repeating | |
|---|---|---|---|---|
| | molecular | | units | |
| | weight (Mn) | | (hydrophilic- | |
| | final polymer* | | cationic- | # of cationic |
| Polymer | (Da) | Polydispersity | hydrophilic) | sites/polymer |
| 422-1 | 13794 | 1.13 | 7-38-7 | 38 |
| 422-2 | 23834 | 1.31 | 14-59-14 | 59 |
| 422-3 | 84605 | 1.75 | 69-113-69 | 113 |
| 1125 | 73088 | 1.72 | 58-108-58 | 108 |
| 1007-1 | 28266 | 1.22 | 15-82-15 | 82 |
| 1007-2 | 39500 | 1.44 | 21-117-21 | 117 |
| 1007-3 | 66630 | 1.85 | 37-192-37 | 192 |

*Molecular weight determined before purification and quaternisation

### Characterization Methods

GPC measurements were performed on a Shimadzu system equipped with a CMB-20A controller system, a SIL-20A HT autosampler, a LC-20AT tandem pump system, a DGU-20A degasser unit, a CTO-20AC column oven, a RDI-10A refractive index detector, and a PL Rapide (Varian) column at 70° C. N,N-Dimethylacetamide (with 2.1 g L⁻¹ of lithium chloride (LiCl)) was used as eluent at a flow rate of 1 mL min⁻¹. The molecular weights reported are relative to polystyrene standards.

¹H-NMR (400 MHz) spectra were recorded using a Bruker Av400 spectrometer at 25° C. either in CDCl₃ or in deuterium oxide (D₂O).

### Example 2

Evaluation of toxicity of the RAFT block copolymers prepared in Example 1 for different cell lines

### Materials

### Cells:

Chinese Hamster Ovary cells constitutively expressing Green Fluorescent Protein (CHO-GFP) (kindly received from K. Wark; CSIRO CMHT Australia) were grown in MEMα modification supplemented with 10% foetal bovine serum, 10 mM Hepes, 0.01% penicillin and 0.01% streptomycin at 37° C. with 5% CO₂ and subcultured twice weekly.

Human embryonic kidney cells (HEK293) cells were grown in RPMI₁₆₄₀ supplemented with 10% foetal bovine serum, 10 mM Hepes, 2 mM glutamine, 0.01% penicillin and 0.01% streptomycin at 37° C. with 5% CO₂ and subcultured twice weekly.

### Toxicity Assay:

CHO-GFP and HEK293 cells were seeded at 3×10⁴ cells per well in 96-well tissue culture plates and grown overnight at 37° C. with 5% CO₂.

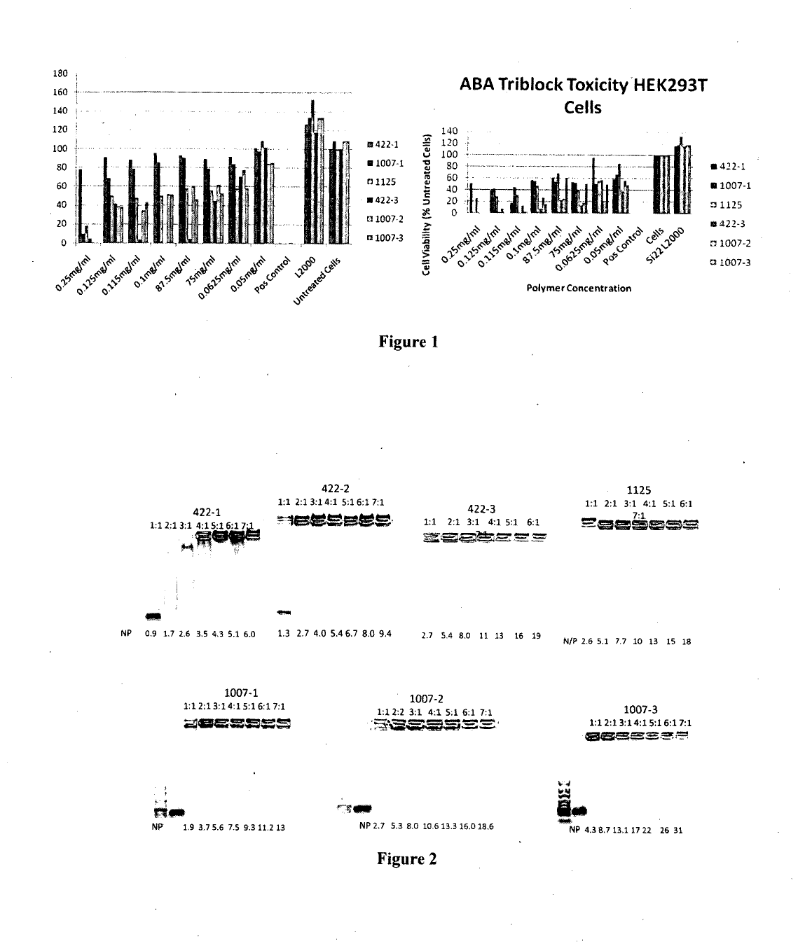

The RAFT block copolymer samples were added to 3 wells in the 96 well culture plates for each sample and incubated for 72 h in 200 μl standard media. Toxicity was measured using the Alamar Blue reagent (Invitrogen USA) according to manufacturer's instructions. Briefly media was removed and replaced with 100 μl of standard media containing 10% Alamar Blue reagent, cells were then incubated for 4 h at 37° C. with 5% CO₂. The assay was read on an EL808 Absorbance microplate reader (BIOTEK, USA) at 540 nm and 620 nm. Cell viability was determined by subtracting the 620 nm measurement from the 540 nm measurement. Results are presented as a percentage of untreated cells. FIG. 1 shows the cell viability results of the block copolymers when tested with CHO-GFP and HEK293T cells.

The toxicity of the polymers was investigated in two cell lines without siRNA association. CHO-GFP cells are a fast growing robust cell line, whilst HEK293T cells are more sensitive to transfection. A range of polymer concentrations were analysed and similar to other findings the more DMAEMA and therefore positive charge the molecule contained a higher apparent toxicity was observed (FIG. 2). An acceptable toxicity level was deemed to be survival of over 60% in both CHO-GFP cells and HEK293T cells. In CHO-GFP cells 422-3 and 1007-2 with similar DMAEMA block lengths were toxic at a concentration of 0.25 mg/ml and became non-toxic at 0.0625 mg/ml whilst 422-1 and 1007-1 were not toxic above 0.25 mg/ml. In HEK293T cells at 0.05 mg/ml 422-1 and 1007-1 were not toxic, however all polymers with a DMAEMA block above a length of 113 (422-3, 1007-2 and 1007-3) were toxic at 0.05 mg/ml in HEK293T cells. 0.05 mg/ml corresponds to a molar ratio of 6:1 of polymer to 50 nM si22, making the standard silencing concentration used non-toxic in CHO-GFP cells but toxic in HEK293T

### Example 3

### Synthetic siRNA and DNA Oligonucleotides

The anti-GFP siRNA was obtained from QIAGEN (USA). The anti-GFP siRNA sequence is sense 5′ gcaagcugacccugaaguucau 3′ (SEQ ID No: l) and antisense 5′ gaacuucagggucagcuugccg 3′ (SEQ ID No:2) and is referred to as si22.

DNA oligonucleotides corresponding to anti-GFP siRNA sequence were purchased from Geneworks (South Australia) and are identified as di22. Oligonucleotides were annealed by combining equal molar amounts of oligonucleotides, heating to 95° C. for 10 min and gradually cooling to room temperature. These were used as negative controls with np silencing effect.

### Formation of Polymer/siRNA Complexes:

Molar ratios of polymer (see Table 1) to 50 nM siRNA or siDNA were calculated. Complexes were formed by the addition of OPTIMEM media (Invitrogen, USA) to eppendorf tubes. The required amount of polymer resuspended in water was added to the tubes and the mixture vortexed. 50 nM of si22 or di22 was then added to the tubes and the sample vortexed. Complexation was allowed to continue for 1 h at RT.

### Agarose Gel Electrophorosis:

Samples at different molar ratios of polymer to 50 nM siRNA were electrophoresed on a 2% agarose gel in TBE at 100V for 40 min. siRNA was visualised by gel red (Jomar Bioscience) on a UV transilluminator with camera, the image was recorded by the GeneSnap program (Syngene, USA).

Previous work has shown that 50 nM of si22 is enough to visualise on an agarose gel and to silence 80% of the EGFP signal in CHO-GFP cells by the control, Lipofectamine 2000 transfection (data not shown). This amount of si22 was therefore used to determine the ability of the polymer to bind the siRNA and to silence EGFP expression in the CHO-GFP cells. Molar ratios of polymer to si22 ranging from 1:1 to 7:1 were formulated for each polymer. This was to ensure a level of polymer below the toxicity limit was used. These samples were subjected to electrophoresis and differences in the ability to associate with the siRNA were observed (FIG. 2: 422-3). siRNA association was determined by the shift of the siRNA from the expected 22 nt migration to being unable to enter the gel to any significant extent. All quasi-ABA triblock polymers with a DMAEMA length of above 59 were able to completely bind the siRNA at a molar ratio of 1:1 or 2:1. 422-1 with the shortest B block length of 38 had the least affinity with the siRNA requiring an N/P ratio of 4.3 corresponding to a molar ratio of 5:1 to show significant complex formation. Interestingly polymers showed different binding affinities even at the same N/P, for example at a N/P ratio of 2.7 422-1 was not able to completely bind the siRNA whilst the majority of the other quasi-ABA triblock polymers were.

The size of the polymer siRNA complexes was determined by DLS (see Example 4)

### Example 4

### Dynamic Light Scattering (DLS) and Zeta Potential Measurements

The hydrodynamic diameters (DH) of siRNA/block copolymer complexes were obtained via dynamic light scattering experiments that employed a Malvern-Zetasizer Nano Series DLS detector with a 22 mW He—Ne laser operating at i) 632.8 nm, an avalanche photodiode detector with high quantum efficiency, and an ALV/LSE-5003 multiple ô digital correlator electronics system. Samples were prepared at a total siRNA concentration of 3500 nM and contained a total mass per volume (i.e., block copolymer mass+siRNA mass per mL) of 0.5 mg/mL while maintaining a N/P ratio of 1.0. To remove dust, samples were centrifuged at 14 000 rpm for 10 min prior to characterization via DLS. All DH measurements were performed in triplicate at 25° C., and complex sizes were compared to those of the uncomplexed block copolymers and the pure siRNA.

Zeta potential were measured in HEPES buffer using automated setting in standard disposable Zeta potential flow cell in the Malvern-Zetasizer. Zeta potential was calculated from the measurement of the mobility of the particles (electrophoretic mobility) in an electrical field and the particle size distribution in the sample.

TABLE 2
Particle size and Zeta potential of complexes formed from RAFT
polymers prepared in Example 1 and siRNA

| | | Zeta Potential | Particle Size |
|---|---|---|---|
| Polymer | Polymer:siRNA | (mV) | (nm)* |
| 422-3 | 3 | 29.1 | 12 ± 0.6 |
| 422-3 | 4 | 38.9 | 16 ± 1.8 |
| 1125 | 3 | 11.3 | 17 ± 3.6 |
| 1125 | 4 | 12.2 | 19 ± 0.1 |
| 1007-2 | 3 | 28.3 | 12 ± 0.2 |
| 1007-2 | 4 | 30.1 | 15 ± 3.4 |
| 1007-3 | 3 | 21.2 | 15 ± 0.6 |
| 1007-3 | 4 | 21.5 | 17 ± 3 |

Notes:
*DLS measurements showed a bimodal particle size distribution and values reported are for smaller size range which constituted nearly 99% of the particles. The particles in the other fraction (~1%) was in the range 93-280 nm

### Example 5

### Silencing Assay

CHO-GFP cells were seeded at 3×10⁴ cells in 96-well tissue culture plates in triplicate and grown overnight at 37° C. with 5% CO₂. For positive and negative controls siRNAs were transfected into cells using Lipofectamine 2000 (Invitrogen, USA) as per manufacturer's instructions. Lipofectamine is the current transfection agent widely used to date and acts as a bench mark in these sets of experiments. Briefly, 50 picomole of the relevant siRNA (corresponding to 250 nM) were mixed with 1 μl of Lipofectamine 2000 both diluted in 50 μl OPTI-MEM (Invitrogen, USA) and incubated at room temperature for 20 mins. The siRNA: lipofectamine mix was added to cells and incubated for 4 h. Cell media was replaced and incubated for 72 h.

For polymer/siRNA complexes prepared according to Example 1 cell media was removed and replaced with 100 μl OPTI-MEM. The polymer/siRNA complexes in a volume of 10 μl was added to 3 wells of cells per sample and incubated for 4 h. Cell media was replaced and cells incubated for a further 72 h.

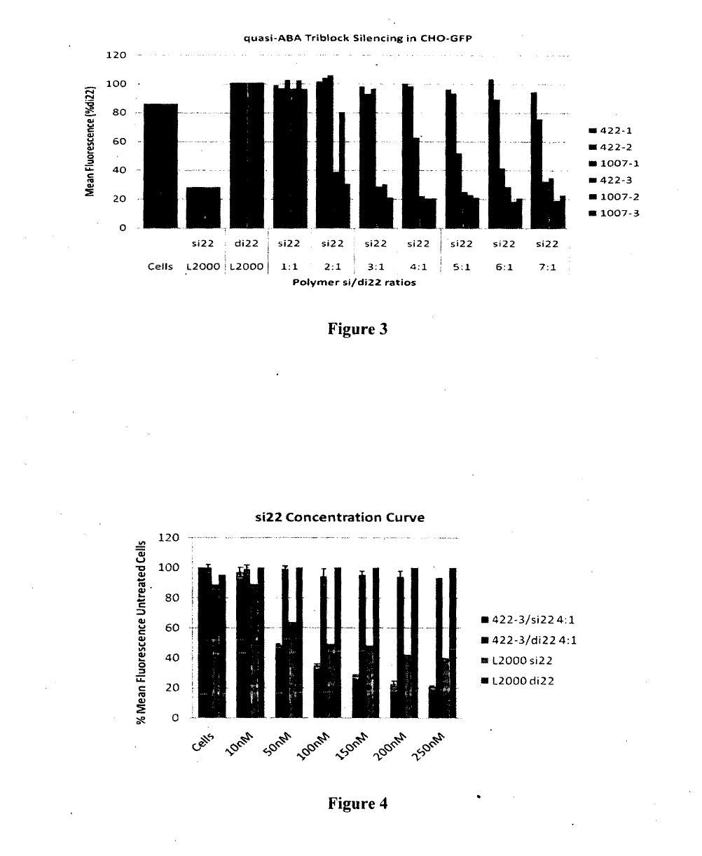

Cells were washed twice with PBS, trypsinised and washed once with FACS wash (PBS with 1% FBS). Cells were subjected to flow cytometry on a Becton Dickenson LSRII and EGFP silencing was analysed as a percentage of the non-silencing siRNA or polymer/siRNA complexes mean EGFP (measured on FITC wavelength) fluorescence. The results are summarized in FIG. 3 and Table 3.

TABLE 3
The N/P ratio, % siRNA binding and silencing efficiency and the polymer
concentration in for various ratios of block copolymer/siRNA complexes

| Molar | Polymer | | | | | | | |
|---|---|---|---|---|---|---|---|---|
| Ratio | 422-1 | | 422-2 | 422-3 | 1125 | 1007-1 | 1007-2 | 1007-3 |
| 1:1 | a) | 0.9 | 1.3 | 2.6 | 2.6 | 1.9 | 2.7 | 4.3 |
| | b) | 10% | 30% | 100% | 96% | 50% | 97% | 100% |
| | c) | 0% | 0% | 0% | 0% | 0% | 0% | 0% |
| | d) | 3.45 | 6.0 | 21.0 | 18.3 | 7.1 | 9.9 | 16.7 |
| 2:1 | a) | 1.7 | 2.7 | 5.4 | 5.1 | 3.7 | 5.3 | 8.7 |
| | b) | 20% | 100% | 100% | 100% | 95% | 100% | 100% |
| | c) | 0% | 0% | 60% | 0% | 0% | 20% | 70% |
| | d) | 6.9 | 11.9 | 42.3 | 36.5 | 14.1 | 19.8 | 33.3 |
| 3:1 | a) | 2.6 | 4.0 | 8.0 | 7.7 | 5.6 | 8.0 | 13.1 |
| | b) | 50% | 100% | 100% | 100% | 100% | 100% | 100% |
| | c) | 0% | 0% | 75% | 20% | 38% | 70% | 79% |
| | d) | 10.35 | 17.9 | 63.5 | 54.8 | 21.2 | 29.6 | 50 |
| 4:1 | a) | 3.5 | 5.4 | 11 | 10 | 7.5 | 10.6 | 17 |
| | b) | 80% | 100% | 100% | 100% | 100% | 100% | 100% |
| | c) | 0% | 5% | 75% | 45% | 48% | 80% | 80% |
| | d) | 13.8 | 23.85 | 84.50 | 73.1 | 28.2 | 39.5 | 66.7 |
| 5:1 | a) | 4.3 | 6.7 | 12.8 | 13 | 9.3 | 13.3 | 22 |
| | b) | 100% | 100% | 100% | 100% | 100% | 100% | 100% |
| | c) | 0% | 0% | 75% | 55% | 51% | 77% | 80% |
| | d) | 17 | 29.8 | 105.5 | 91.3 | 35.3 | 49.4 | 83.3 |
| 6:1 | a) | 5.1 | 8.0 | 16 | 15.0 | 11.2 | 16.0 | 26 |
| | b) | 100% | 100% | 100% | 100% | 100% | 100% | 100% |
| | c) | 5% | 0% | 60% | 60% | 64% | 82% | 80% |
| | d) | 20.5 | 35.8 | 127 | 109.6 | 42.4 | 59.3 | 100 |
| 7:1 | a) | 6.0 | 9.4 | 19 | 18 | 13 | 18.6 | 31 |
| | b) | 100% | 100% | 100% | 100% | 100% | 100% | 100% |
| | c) | 5% | 30% | 60% | 60% | 70% | 81% | 80% |
| | d) | 24 | 41.7 | 148 | 127.9 | 49.4 | 69.2 | 116.7 |

a) N/P ratio;
b) % siRNA binding;
c) % silencing efficiency;
d) polymer concentration μg/mL. Shading indicates toxic concentration of polymer without siRNA

CHO-GFP cells ubiquitously express enhanced green fluorescent protein which when excited by a blue 408 nm laser emits a green signal at approximately 518 nm. This is readily detected by both fluorescence microscopy and flow cytometry. Silencing of the EGFP is therefore easily determined by a shift in the cell population on a flow cytometry plot and by a decrease in mean GFP fluorescence. Addition of the polymers at the range of molar ratios showed that 422-3, 1007-2 and 1007-3 at a molar ratio of 3:1 and above corresponding to an N/P ratio of 8 and above were able to show a significant level of silencing (FIGS. 3 and 4). The Zeta potential of 1125 is lower than that of 422-3.

To determine the minimum concentration of siRNA in the 422-3 sample required to give equivalent silencing to Lipofectamine 2000, a dilution curve was performed using Lipofectamine 2000 and 422-3 at a 4:1 ratio. The minimum amount of siRNA required to achieve equivalent silencing and therefore significant effect was 100 nM.

### Example 6

### Serum Stability

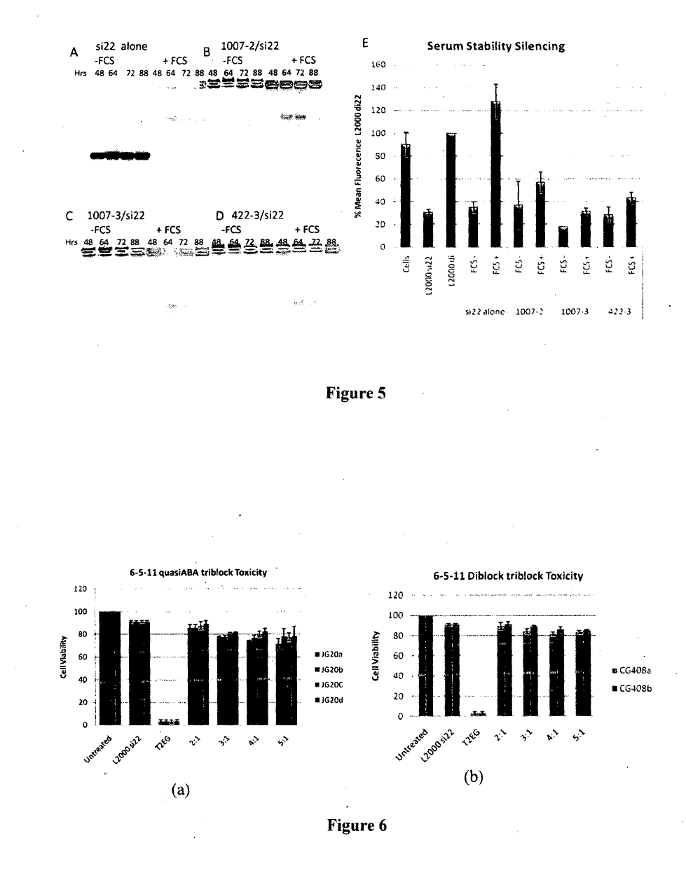

The ability of the polymer to protect the siRNA from degradation by serum proteases was performed in vitro using foetal bovine serum which is commonly used in tissue culture to provide essential growth hormones. Whilst naked siRNA is degraded in this serum within a few hours, the results show that the siRNA contained with in the polymer complexes was protected for up to 88 hours at 37° C. (FIG. 5). The remaining samples were then added to CHO-GFP cells to determine if the siRNA was intact and still active. Silencing was observed with all polymer complexes with little decrease in activity after FBS treatment (FIG. 5E). No precipitation of the complexes was observed with the serum which is also a concern as positively charged molecules are known to associate with serum proteins and precipitate out of solution (data not shown).

### Example 7

In this Example, a series of quasi-triblock copolymers were prepared to systematically evaluate the effect of having small amounts of cationic monomer DMAEMA in the hydrophilic block PEGMA on toxicity, siRNA uptake and gene silencing.

Methods described in Example 1 were used to purify monomers and to prepare the bis chain transfer agent (I) to synthesize polymers in this Example.

### Step 1: Synthesis and Characterization of PDMAEMA Telechelic macroRAFT Agent

DMAEMA monomer (3.15 g, 2.00×10⁻² mol), VAZO-88 initiator (2.64 mg, 1.08×10⁻⁵ mol), the bis-RAFT agent (I) (480 μL of 0.328 g/mL stock solution in DMF, 1.8×10⁻⁷ mol) and DMF (2.02 g, 2.76×10⁻² mol) were dispensed into a glass vial and mixed until all components were dissolved. The reaction mixture was then transferred into a Young vessel containing a magnetic stirrer and subjected to three freeze-pump-thaw cycles between −78° C. and room temperature. Thereafter, the reaction mixture was heated up to 90° C. for 12.5 h. The obtained monomer to polymer conversion was 91% as determined by ¹H-NMR (as explained in Example 1, Step 1).

The obtained reaction mixture was diluted with DCM and the polymer was precipitated by adding the DCM mixture drop-wise into n-heptane. The supernatant was decanted from the polymer residue and this precipitation procedure was carried out a further two times. The polymer was then dried under vacuum at 40° C. until a constant weight was reached. The Mn of the polymer was estimated to be 23635 Da (PDI of 1.15) as determined by GPC against polystyrene standards.

### Step 2: Synthesis and Characterization of P(OEGMA₄₇₅-b-DMAEMA-b-OEGMA₄₇₅) with Variable Incorporation of DMAEMA Monomer Units into the P(OEGMA₄₇₅)-Blocks

Four polymer variants were prepared differing in their quantity of DMAEMA. DMAEMA monomer units were incorporated into the P(OEGMA₄₇₅) blocks at the level of 0, 2, 5 and 10 mol % with respect to the DMAEMA starting material used for the original PDMAEMA precursor telechelic macroRAFT agent synthesis.

A reagent solution common to the synthesis of each of four variants was prepared. The dried PDMAEMA homopolymer (telechelic macroRAFT agent) (1.40 g, 1.45×10⁻² mol) was redissolved in DMF (4.43 g, 2.06×10⁻² mol) and to this solution was added OEGMA₄₇₅ monomer (1.70 g, 3.58×10⁻³ mol), VAZO-88 (initiator) dissolved in DMF (0.1 ml of 1.2 mg/mL solution, 4.90×10⁻⁴ mol) and trioxane (45 mg, 5.00×10⁻⁴ mol). This reagent solution was stirred until all components were dissolved and split into four aliquots (4×1.895 g reaction mixtures). DMAEMA monomer in DMF (39.4 mg/mL) and DMF solvent was added in the volumes shown Table 4.

TABLE 4
DMAEMA and solvent addition for each reaction variant.

| | | Volume in mL | DMF |
|---|---|---|---|
| | Mol % | of DMAEMA in DMF | added |
| Reaction | DMAEMA | solution (39.4 mg/mL) | mL |
| 189JG14A | 0 | 0 | 1.17 |
| 189JG14B | 2 | 0.20 | 0.97 |
| 189JG14C | 5 | 0.50 | 0.67 |
| 189JG14D | 10 | 1.00 | 0.17 |

The reaction mixtures in all cases (A-D) were heated up to 90° C. for 6 h. The resultant monomer to polymer conversion was determined (by ¹H-NMR) to be in the range of 86-88% for each reaction (in CDCl₃; following a similar procedure as explained above for the polymerization of DMAEMA). The Mn and PDI were determined by GPC against PS standards. The values of these parameters are shown in Table 5.

TABLE 5
¹H NMR based conversion and GPC based Mn and PDI data for
DMAEMA variant products of the reactions 189JG014A-D.

| Reaction | Mol % | % Conversion | |
|---|---|---|---|
| Product | DMAEMA | (¹H NMR) | Mn (GPC) |
| 189JG14A | 0 | 86 | 38463 |
| 189JG14B | 2 | 88 | 39642 |
| 189JG14C | 5 | 88 | 39846 |
| 189JG14D | 10 | 86 | 41017 |

### Purification of Triblock Copolymers:

The polymerized reaction mixtures from Step 1 (198JG14A-D) were separately diluted with DCM and each polymer was precipitated by adding the mixture drop-wise into diisopropyl ether; the precipitated polymer was decanted from the rest of the solution. This later procedure was carried out two times. In a final step the polymer was dried under vacuum until constant weight was reached. Further purification of the polymeric material was carried out by dialysis (molecular weight cut-off of 3500, Spectra Por, Spectrum Medical Industries, Inc., Houston, Tx) against Milli-Q water for 3 days. After dialysis, the water was removed from the aqueous polymer solution by use of a Rotovapor Evaporator.

### Quaternisation:

The polymers were redissolved in DCM and an excess of methyl iodide was added into these solutions in the volumes specified in Table 6 below. The reactions were stirred for 2 h at room temperature in order to achieve the quaternisation of the tertiary amino groups of the DMAEMA groups incorporated into the quasi-triblock copolymers.

TABLE 6
Volume of methyl iodide and DCM used for the quarterisation
of quasi-triblock copolymers.

| | unquartanised | Mass of | | Volume |
|---|---|---|---|---|
| | Starting | Starting | Volume DCM | methyl |
| Reaction | Material | Material (g) | (mL) | iodide (mL) |
| 189JG18A | 189JG14A | 0.389 | 3.9 | 0.65 |
| 189JG18B | 189JG14B | 0.526 | 5.3 | 0.88 |
| 189JG18C | 189JG14C | 0.497 | 5.0 | 0.83 |
| 189JG18D | 189JG14D | 0.570 | 5.7 | 0.95 |

The solvent and excess of methyl iodide were removed in a Rotovapor Evaporator and the polymer was dried under vacuum at 40° C. for 24 h.

Copolymers were characterised by GPC and NMR as described in Example 1 and the results are summarized in Table 7.

TABLE 7
Molecular weight, block copolymer composition and DMAEMA
content in OEGMA₄₇₅ block of the triblock copolymer

| Polymer | | Composition: # of repeat | % DMAEMA in |
|---|---|---|---|
| Samples | Mn | units in each block | OEGMA₄₇₅ block |
| 189JG020A | 38298 | 15-145-15 | with 0% DMAEMA |
| 189JG020B | 39624 | 17-145-17 | with 2% DMAEMA |
| 189JG020C | 40460 | 18-145-18 | with 5% DMAEMA |
| 189JG020D | 41526 | 19-145-19 | with 10% DMAEMA |

The copolymers prepared in this example exhibited similar molecular weights as expected. The central cationic block in these copolymers has identical block length and similar OEGMA₄₇₅ blocks with DMAEMA monomer units in the range 0 to 10%. With increasing DMAEMA content the polymer molecular weight increased accordingly.

### Example 8

This example illustrates the preparation of diblock copolymers from DMAEMA and OEGMA₄₇₅ to compare with the triblock copolymers prepared in Example 1 (method B) and Example 6.

Procedures described in Example I were used to purify monomers and the chain transfer agent (II) was synthesized and purified as described below.

RAFT Agent: 4-Cyano-4-[(dodecylsulfanylthiocarbonyl)sulfanyl]pentanoic acid (II): C₁₉H₃₃NO₂S₃; MW 403.17

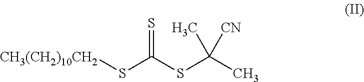

was synthesized following the procedure described in WO 2005/113493 A1.

### Step 1: Synthesis and Characterization of PDMAEMA macroRAFT Agent

In a typical polymerization experiment, 2358.15 mg of DMAEMA monomer (1.50×10⁻² mol), 1.98 mg of VAZO-88 initiator (8.10×10⁻⁶ mol), and 32.70 mg of RAFT agent (8.10×10⁻⁵ mol) and 4694.05 mg of DMF (6.42×10⁻² mol) were mixed together in a Young vessel containing a magnetic stirrer. The reaction mixture was degassed by sparging nitrogen gas for at least 15 min and subjected to three freeze-pump-thaw cycles between −78° C. and room temperature.

Thereafter, the reaction mixture was heated up to 90° C. for 1 h. The monomer to polymer conversion achieved was 44% as determined by ¹H-NMR (as explained in Example 1, Step 1). The Mn of the polymer was 20187 Da (PDI of 1.32) as determined by GPC against polystyrene standards.

### Step 2: Synthesis and characterization of Poly(oligo(ethylene glycol) methyl ether methacrylate-block-N,N-Dimethylaminoethyl methacrylate (P(OEGMA₄₇₅-b-DMAEMA)

The reaction mixture from the polymerization in step 1 was diluted with DCM and the polymer was precipitated by adding drop-wise the mixture into n-heptane; the precipitated polymer was decanted from the rest of the solution. This later procedure was carried out two times. In a final step the polymer was dried under vacuum at 40° C. until constant weight. 487.2 mg of dried polymer (PDMAEMA homopolymer (macroRAFT agent), 2.41×10⁻⁵ mol) were redissolved in 5600 mg of DMF (7.66×10⁻² mol) in a Young vessel containing a magnetic stirrer. 1.3 mg of VAZO-88 (initiator, 5.30×10⁻⁶ mol) dissolved in 1910 mg of OEGMA₄₇₅ (monomer, 4.02×10⁻³ mol) were added into this later solution and exposed to the degassing method abovementioned for the case of the synthesis of PDMAEMA macroRAFT agent. Thereafter, the reaction mixture was heated up to 90° C. for 3 h. The materials obtained from this approach are expected to have a macromolecular architecture known as diblock copolymer (since residual monomer of polymerization in step 1 was removed by the explained precipitation procedure). Polymer 0408-A was prepared using this method. Polymer sample 0408-B was prepared using a similar methods as described above but using 379.3 mg of dried polymer (PDMAEMA homopolymer (macroRAFT agent), 1.87×10⁻⁵ mol) redissolved in 2866 mg of DMF (3.92×10⁻² mol) and 0.66 mg of VAZO-88 (initiator, 2.70×10⁻⁶ mol) dissolved in 950 mg of OEGMA₄₇₅ (monomer, 2.00×10⁻³ mol). This later reaction mixture was heated up to 90° C. for 2 h. Table 8 summarizes the properties of the diblock copolymers synthesized using these latter methods.

### Purification of Diblock Copolymers:

In both cases, the obtained reaction mixtures of block copolymers were diluted with DCM and the polymer was precipitated by adding drop-wise the mixture into diisopropyl ether; the precipitated polymer was decanted from the rest of the solution. This later procedure was carried out two times. In a final step the polymer was dried under vacuum until constant weight. Further purification of the polymeric material was carried out by dialysis (molecular weight cut-off of 3500, Spectra Por, Spectrum Medical Industries, Inc., Houston, Tx) against MiliQ water for 3 days. After dialysis, the water was removed aqueous polymer solution in a Rotovapor Evaporator.

### Quaternisation:

The polymer was redissolved in DCM and an excess of methyl iodide was added into this solution and stirred for 2 h at room temperature in order to achieve the quaternization of the tertiary amino groups of PDMAEMA block of the diblock copolymer. In a final step, DCM and excess of methyl iodide were removed in a Rotovapor Evaporator; the polymer was further dried under vacuum at 40° C. for 24 h.

TABLE 8
AB block copolymers prepared in Example 1

| | Experimental | | | |
|---|---|---|---|---|
| | molecular | | # of repeating | |
| | weight (Mn) | | units | |
| | final | | (hydrophilic- | # of cationic |
| Polymer | polymer (Da) | Polydispersity | cationic) | sites/polymer |
| 0408-A | 43313 | 2.20 | 47-126 | 126 |
| 0408-B | 33298 | 1.50 | 28-126 | 126 |

### Example 9

The cell viability, or cell toxicity, results in FIG. 6 show both tri and di block copolymers prepared in Examples 7 and 8 have no significant effect on the cells within the concentration ratios of polymer to siRNA investigated. The polymer T2EG is a polymer known to have very poor cell viability and used as a positive control. These results confirm that the polymers are suitable as delivery vehicles and in that they are not toxic at a wide range of concentrations.

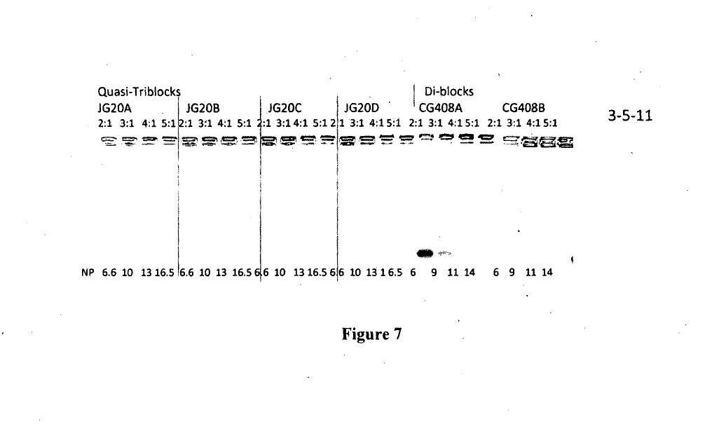

The stability of the polymer-siRNA complex in in vivo conditions (ie in serum) is measured by the size degradation or lack thereof of the complexes and how they move through an electrophoresis size exclusion gel, as illustrated in FIG. 7. All polymers in the triblock series exhibited strong binding to siRNA as the complexes showed no signs of dissociation under the experimental conditions used. However, the diblock copolymers showed relatively poor binding, particularly for 0408A, and furthermore the binding was weaker for compositions with lower ratios of polymer to siRNA.

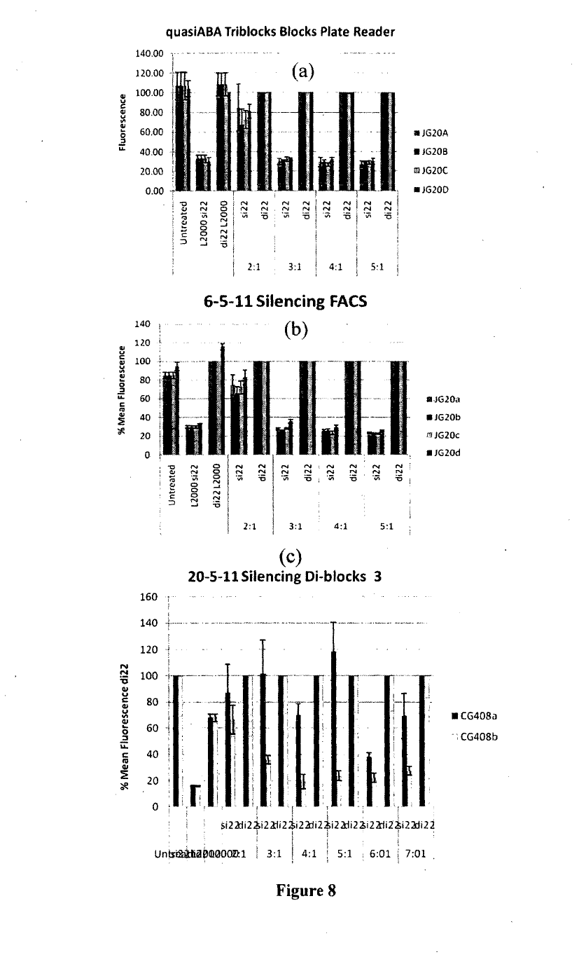

The relative silencing efficacy of various copolymers in both triblock and diblock series is compared in FIG. 8. This figure shows that triblocks silence the gene, as evidenced by no fluorescence.

Taken together these comparative results demonstrate that whilst diblocks are not toxic to a variety of cells and only some diblocks demonstrate good stability it was only the triblocks that showed gene silencing.

### Example 10

### Chain Extension of Polymer Sample 1007-2 with PolyFluor® 570 (1007-2/PF)

In a Young vessel, a sample of 1007-2 polymer (from Example 1, see. Table 1 for details) (147 mg), methacryloxyethyl thiocarbamoyl Rhodamine B (PolyFluor®570, Polysciences, Inc.) (6.8 mg), and AIBN (1.0 mg) were dissolved in DMF (2 mL). The mixture was degassed by three freeze-evacuate-thaw cycles under high vacuum (3×10⁻³ Torr) and then heated at 60° C. for 21 hours. Solvent (DMF) was removed and the sample was dried under vacuo, yielding final polymer 1007-2/PF in quantitative yield, and the chemical structure is shown below. Molecular weight of 1007-2/PF can't be determined accurately by GPC due to the polymer sample being quaternised already making it undetectable by refractive index detector. The final pure polymer was obtained after dialysis with Milli-Q water for 3 days. After dialysis, the water was removed from the aqueous polymer solution by freeze drying. The dialyzed polymer was analysed according to the procedures described in the following section.

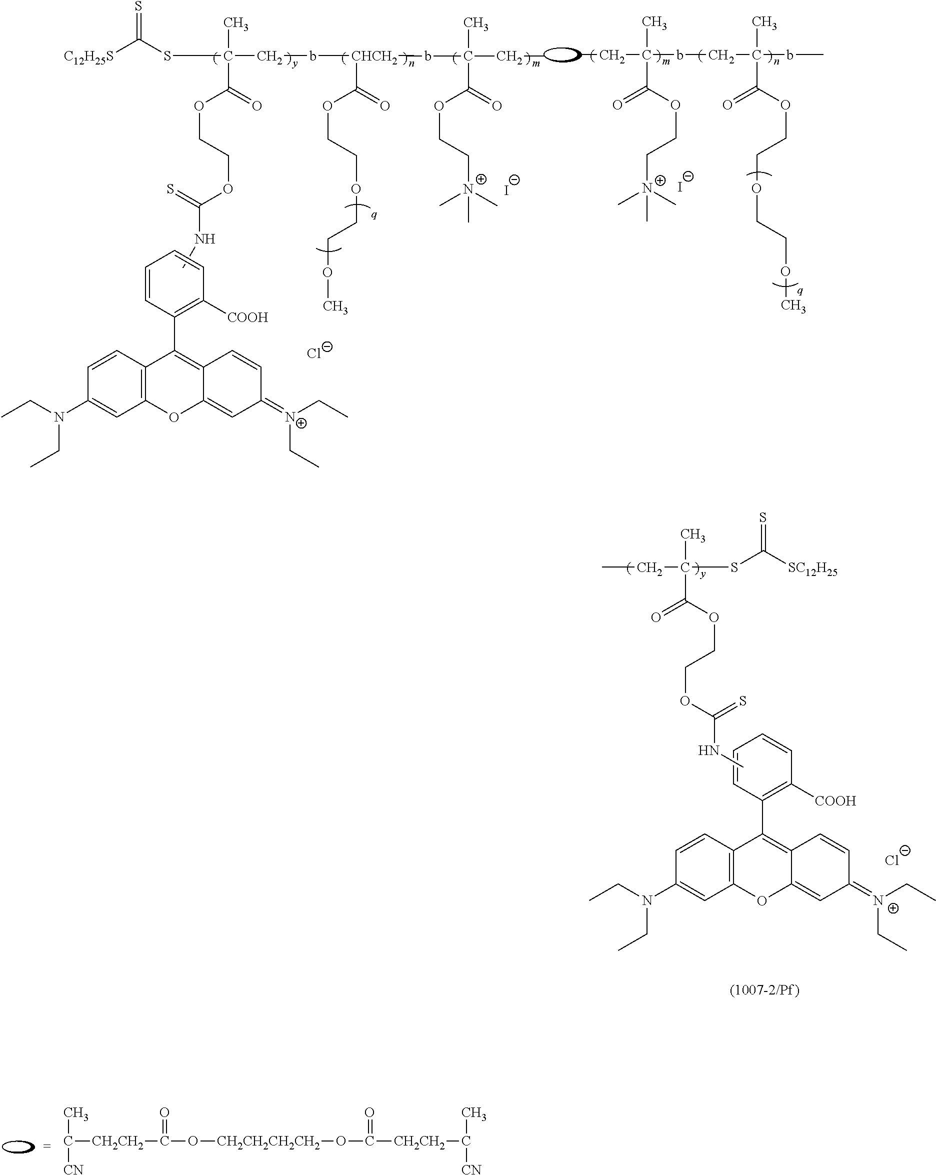

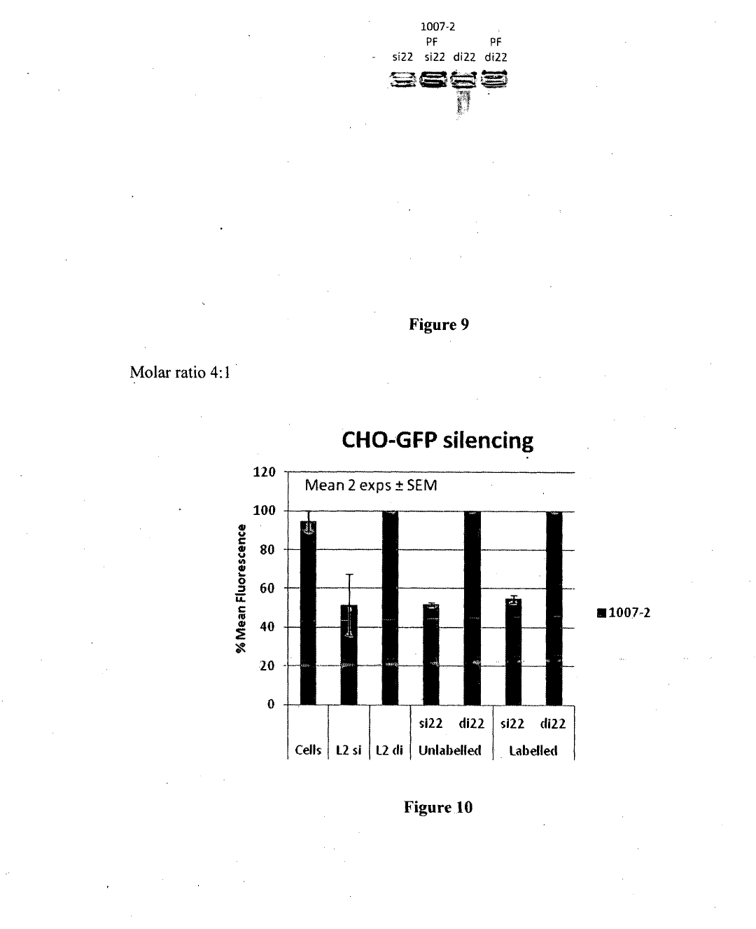

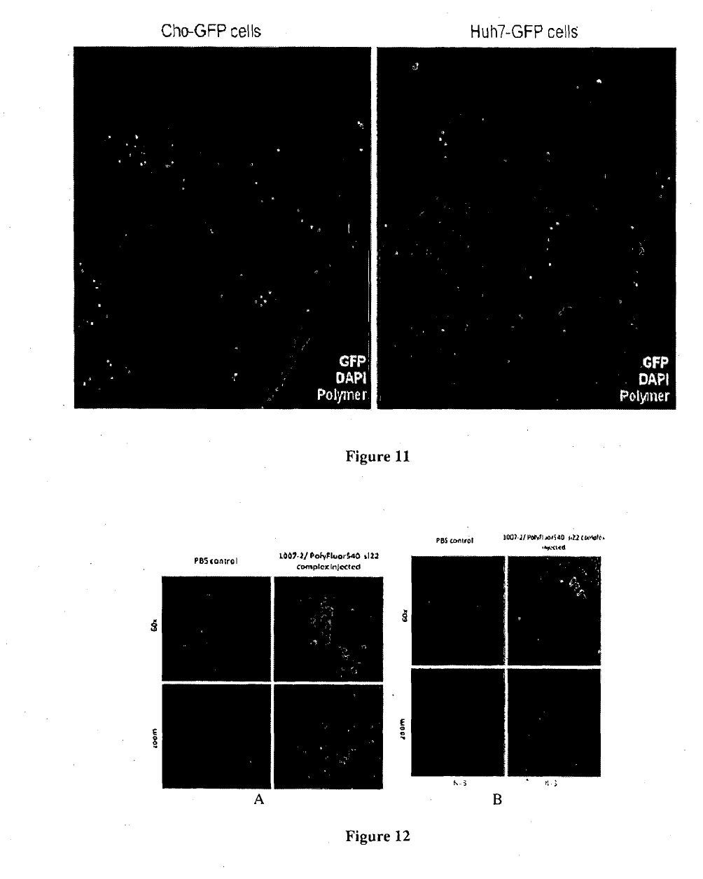

The biological evaluation of 1007-2/PF was carried out according to the test protocols described in Example 2 and 5. The siRNA binding was evaluated by electrophoresis as described in Example 2 and the results are illustrated in FIG. 9. The silencing of CHO-GFP was evaluated by using the method described in Example 5. FIG. 10 illustrates the comparative silencing of polymer with and without labelling with PolyFluor. The cellular uptake of labelled polymer (1007-2/PF) and siRNA was further illustrated by confocal microscopy; FIG. 11 illustrates the uptake of labelled polymer by CHO-GFP and Huh-GFP cells.

### Example 11

The polymer (1007-2/PF) prepared in Example 10 was used for all biological evaluations described in this Example.

### IFN Response In Vivo:

Commercial day 10 chicken embryos were obtained from Charles River Laboratories, Australia. Polymer complexes were injected into the allantoic cavity of a 10-day-embryonated chicken egg. The eggs were incubated at 37° C. for 6 or 24 h. PBS and si22 alone at 2 nmole were injected into eggs as controls. Allantoic membrane and liver were collected into RNA later and stored at 4° C. RNA was harvested using the Trizol method (Chomczynski and Sacchi 1987).

### Histopathology and Allantoic Membrane Uptake:

Embryonic chicken livers were obtained from the same embryos as the membrane studied for IFN response at 24 h. Livers were fixed in 10% buffered formalin for 24 h and submitted to the pathology laboratory at the Australian Animal Health Laboratories for routine H&E staining. Allantoic membranes were fixed in 4% paraformaldehyde for 2 h. Membranes were then permeabilized for 1 h in PBS plus 0.1% Triton X-100, and stained with DAPI for 20 min to visualize nuclei.

### Reverse Transcription and Quantitative Real-Time PCR:

One microgram of extracted RNA was treated with DNase (Promega, USA) according to manufacturer's instructions, quantitative real-time PCR (QRT-PCR) experiments were conducted using power Sybr green RNA to CT kit (Applied Biosystems, USA) according to manufacturer's instructions to measure cytokine expression levels. All quantification data was normalised against chicken or human GAPDH. QRT-PCR was performed on a StepOnePlus Real Time-PCR System, 96 well plate RT-PCR instrument (Applied Biosystems) under the following conditions: 1× cycle 50° C. for 30 minutes followed by 95° C. for 10 minutes, 40× cycles 95° C. for 15 seconds followed by 60° C. for 1 minute. The comparative threshold cycle (Ct) method was used to derive fold change gene expression.

Chicken qRT-PCR primer sequences have been published previously (Karpala, Lowenthal et al. 2008), human qRT-PCR primer sequences were obtained from qPrimer Depot (http://primerdepot.nci.nih.gov/). Primers were obtained from Geneworks (Sth Australia).

### In Vivo Influenza A-PR8 Silencing:

Commercial day 10 chicken embryos were obtained from AAHL small animal facility. Polymer complexes were injected into the allantoic cavity of a 10-day-embryonated chicken egg. The eggs were incubated at 37° C. for 24 h. PBS was injected as a control. H1N1 Influenza PR8 virus was diluted in 100 μl PBS to 500 pfu/egg and immediately injected into the allantoic cavity of a 10-day-embryonated chicken egg. The eggs were incubated at 37° C. for 48 h and allantoic fluid was harvested to measure virus titre.

### Influenza Assays:

TCID₅₀ assays were performed as described in (Liang, Mozdzanowska et al. 1994). Briefly, tissue culture supernatants or allantoic fluid were assayed for virus infectivity on MDCK cells by endpoint dilution for cytopathic effect with a 10-fold dilution series. Titres are expressed as log 10 TCID₅₀/ml±SEM.

### Results

Uptake of 1007-2/PF si22 Complexes In Vivo:

This is a widely used model of influenza infection. The main site of replication of influenza in eggs is the allantoic membrane. To determine if we could deliver 1007-2 siRNA complexes to the allantoic membrane 10 day old embryonated chicken eggs were injected with 1007-2/PF into the allantoic fluid. This polymer is 1007-2 with a Polyfluor 540 monomer extension. This monomer fluoresces when excited by a 540 nm laser and emits at 590 nm. Allantoic membrane was removed at 6 or 24 h and polymer was visualised by confocal microscopy. Polymer was clearly visible in cells associated with veins in the allantoic membrane at 6 h (FIG. 12A) and had disseminated throughout the membrane by 24 h (FIG. 12B). This indicates siRNA would be present in the majority of the membrane cells when PR8 virus was injected

### Toxicity of 1007-2 In Vivo:

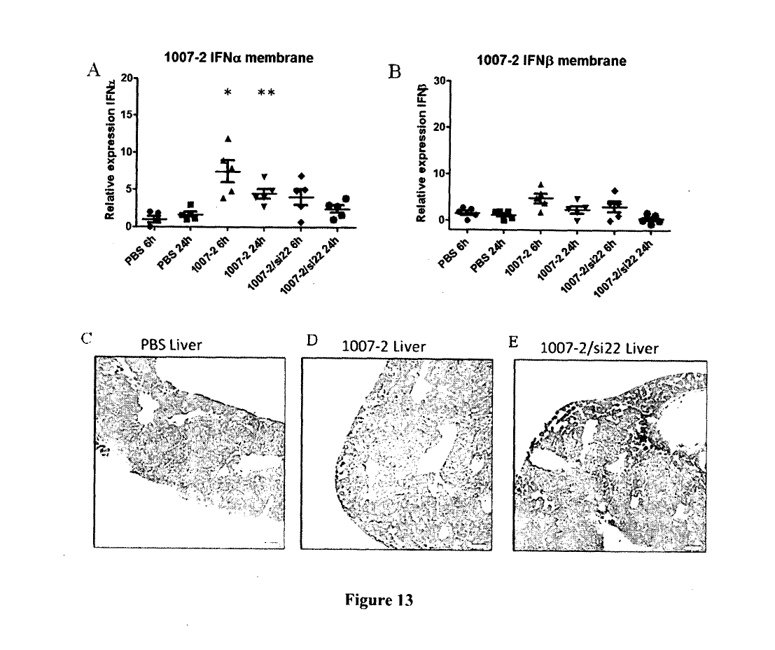

The earlier in vitro results with the polymer alone and polymer si22 complexes are herein confirmed by injection into the embryos and assayed for IFNα and β induction and PR8 silencing. An average 8 fold IFNα induction at 6 h and 5 fold induction at 24 h was observed in polymer alone treated embryos, compared to 5 and 3 fold in the polymer/si22 treated embryos (FIG. 13A). This result supports the in vitro findings. No significant IFNβ was induced in the allantoic membrane although a similar pattern to IFNα was observed (FIG. 13B). Histopathology on 3 embryonic chicken livers per group performed by the pathology laboratory at Australian Animal Health Laboratory showed there was no clinical signs of damage to the livers at 24 h indicating minimal toxicity to the embryos, representative figures are shown (FIGS. 13C, D & E).

PR8 Silencing to 1007-2 In Vivo:

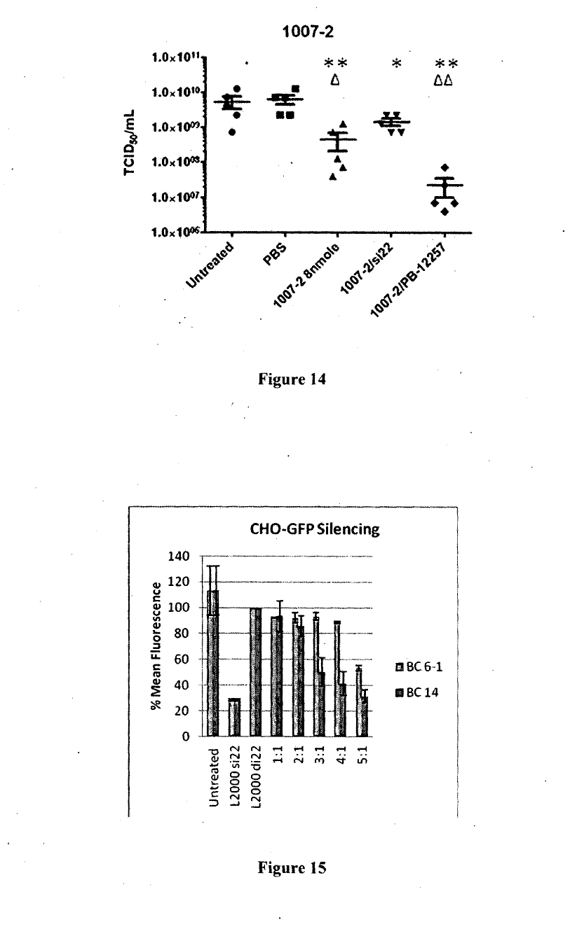

Uptake of the polymer siRNA complexes in the allantoic membrane was observed by confocal microscopy at the time of virus injection. As expected when PB1-2257 was delivered to the chicken embryos an average 2.5 log decrease was observed compared to PBS treated embryos and a 1.5 log decrease in virus replication when compared to the polymer/si22 treated embryos (FIG. 14). This indicates delivery of the siRNA to the embryo and specific silencing. A 1 log decrease was observed with 1007-2 alone; this again supports the in vitro findings and indicates the induction of IFNα by the polymer may be resulting in non-specific IFN induced virus reduction. A 0.5 log reduction of productive virus was seen with 1007-2/si22, this decrease in virus is presumably due to the minor IFNα induction seen with this complex.

### Example 12

### ABA Triblock Copolymers Containing 4-Vinylphenylboronic Acid (VPBA) or 4-Vinylphenylboronic Acid Pinacol Ester (VPBA-PE)

In this Example, ABA triblock copolymers containing 4-vinylphenylboronic acid (VPBA) or 4-vinylphenylboronic acid pinacol ester (VPBA-PE) with PolyFluor® 570 were prepared in order to evaluate the effect of RAFT polymers having boronic acid functionality in the hydrophilic block POEGMA₄₇₅ on toxicity, siRNA uptake, cell targeting and gene silencing.

The bis-RAFT agent (I) was used to synthesize these ABA triblock copolymers in this Example.

### Step 1: Synthesis of Mid Block PDMAEMA Telechelic macroRAFT Agent

DMAEMA monomer (7.86 g, 4.99×10⁻² mol), VAZO-88 initiator (6.6 mg, 2.68×10⁻⁵ mol), the bis-RAFT agent (I) (0.359 g, 4.17×10⁻⁴ mol) and DMF (12.34 g, 16.88×10⁻² mol) were transferred into a Young vessel and subjected to three freeze-pump-thaw cycles between liquid nitrogen temperature and room temperature. Thereafter, the reaction mixture was heated at 80° C. for 16 hours and then heated at 90° C. for additional 16 hours. The obtained monomer to polymer conversion was greater than 95% as determined by ¹H-NMR.

The polymerisation mixture above was diluted with DCM and the polymer was precipitated by adding the DCM mixture drop-wise into n-heptane. The supernatant was decanted from the polymer residue and this precipitation procedure was carried out a further two times. The polymer was then dried under vacuum until a constant weight was reached. The Mₙ of the polymer was determined to be 18,630 Da (PDI of 1.1) by GPC (using N,N-dimethylacetamide as eluent) against polystyrene standards. This molecular weight corresponds to 110 cationic units in the polymer PDMAEMA formed.

### Step 2: Synthesis and Characterization of ABA Triblock Copolymers Containing VPBA or VPBA-PE with PolyFluor®570 in the A Block

Three triblock copolymers P[(OEGMA₄₇₅-co-VPBA-co-PolyFluor® 570)-b-PDMAEMA-b-(OEGMA₄₇₅-co-VPBA-co-PolyFluor® 570)], namely, samples BC-11; BC-13-1 and BC-13-2 were synthesised with various molar amounts used of OEGMA₄₇₅, VPBA and PolyFluor® 570 respectively, and a triblock copolymer of P[(OEGMA₄₇₅-co-(VPBA-PE)-co-PolyFluor® 570)-b-PDMAEMA-b-(OEGMA₄₇₅-co-(VPBA-PE)-co-PolyFluor® 570)] was also synthesised as following.

### Synthesis of BC-11

The dried PDMAEMA telechelic macroRAFT agent from STEP 1 (0.508 g) was redissolved in DMF (14 mL) and to this solution was added OEGMA₄₇₅ monomer (2.50 g, 5.263×10⁻³ mol), 4-vinylphenyl boronic acid (VPBA, 0.16 g, 1.081×10⁻³ mol), AIBN initiator (2.5 mg, 1.52×10⁻⁵ mol) and PolyFluor® 570 (21 mg, 3.07×10⁻⁵ mol)). This reagent solution was then transferred into a glass ampoule. The ampoule and its contents were then degassed by three repeated freeze-evacuate-thaw cycles and then flame sealed. The polymerisation was carried out at 60° C. for 16 hours. Solvent (DMF) was removed on rotary evaporator under vacuum to give a thick slurry.

The polymerisation mixture above was diluted with dichloromethane and the polymer was precipitated by adding the mixture drop-wise into diisopropyl ether; the precipitated polymer was decanted from the rest of the solution. This procedure was carried out two more times to ensure the un-reacted OEGMA₄₇₅ monomer being removed completely. In a final step the polymer was dried under vacuum until constant weight was reached, gave 1.24 g polymer sample BC-11. The Mₙ of the polymer was determined to be 86,900 Da (PDI of 1.53) by GPC (using N,N-dimethylacetamide as eluent) against polystyrene standards.

### Quaternisation:

In a round bottom flask, the above polymer BC-11 (510 mg) was dissolved in acetonitrile (10 mL) and an excess of methyl iodide (2 mL) was added into this solution. The reaction was stirred at room temperature overnight in order to achieve the quaternisation of the tertiary amino groups of the DMAEMA groups incorporated into the triblock copolymers. After removal of solvent (acetonitrile) to dryness, the quaternised ABA triblock polymer was obtained and subjected to the final purification step of dialysis (molecular weight cut-off of 3500, Spectra Por, Spectrum Medical Industries, Inc., Houston, Tx) against Milli-Q water for 3 days. After dialysis, the water was removed from the aqueous polymer solution by use of a freeze dryer to give polymer sample of BC-12.

### Structure of Polymer Sample BC-12

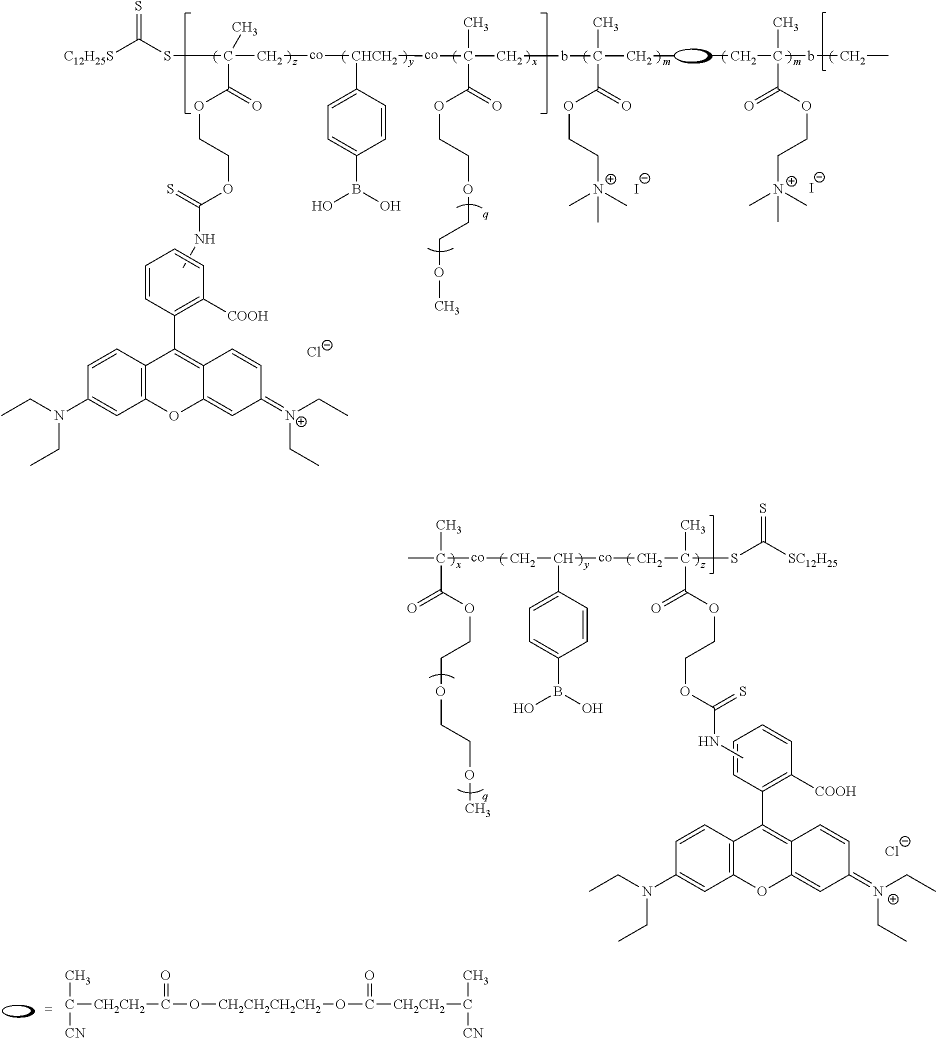

### Syntheses of BC-13-1 and BC-13-2

These two polymer samples were prepared similarly as described above for BC-11 with the exception of various molar amounts of OEGMA₄₇₅ and VPBA (see Table below) were used in the polymerisations.

| | OEGMA475 | VPBA | PolyFluor^{R} 570 |
|---|---|---|---|
| BC-11 | 5.263 × 10⁻³ | 1.081 × 10⁻³ | 3.07 × 10⁻⁵ |
| BC-13-1 | 4.189 × 10⁻³ | 2.162 × 10⁻³ | 3.07 × 10⁻⁵ |
| BC-13-2 | 4.715 × 10⁻³ | 1.621 × 10⁻³ | 3.07 × 10⁻⁵ |

The Mₙ of these two polymers of BC-13-1 and BC-13-2 were determined to be 92,600 and 98,900, respectively by GPC (DMAc as eluent) against polystyrene standards.

### BC-14: ABA triblock copolymer containing 4-vinylphenyl boronic acid (VPBA) and D-(+)-Galactose

A 1:1 molar ratio of the above polymer sample BC-12 (88.7 mg) and D-(+)-Galactose (3.87 mg) were dissolved in deuterated dimethyl sulfoxide (DMSO-d₆) at room temperature for five days. Water (2 mL) was added and the resultant polymer was subjected to dialysis to remove any un-binding Galactose. After freeze dryer, the final polymer BC-14 was obtained and its proton NMR (in D₂O) revealed the presence of Galactose sugar peaks.

### Synthesis of BC-6-1

The PDMAEMA telechelic macroRAFT agent from STEP 1 (0.508 g) was redissolved in DMF (10 mL) and to this solution was added OEGMA₄₇₅ monomer (2.50 g, 5.263×10⁻³ mol), 4-vinylphenylboronic acid pinacol ester* (VPBA-PE, 0.25 g, 1.087×10⁻³ mol), AIBN initiator (2.5 mg, 1.52×10⁻⁵ mol) and PolyFluor®570 (21 mg, 3.07×10⁻⁵ mol)). This reagent solution was then transferred into a glass ampoule. The ampoule and its contents were then degassed by three repeated freeze-evacuate-thaw cycles and then flame sealed. The polymerisation was carried out at 60° C. for 16 hours. Solvent (DMF) was removed on rotary evaporator under vacuum to give a syrupy polymer.

The polymerisation mixture above was diluted with dichloromethane and the polymer was precipitated by adding the mixture drop-wise into diisopropyl ether; the precipitated polymer was decanted from the rest of the solution. This procedure was carried out two more times to ensure the un-reacted OEGMA₄₇₅ monomer being removed completely. In a final step the polymer was dried under vacuum until constant weight was reached, gave 1.08 g polymer sample BC-6-1. The Mₙ of the polymer was determined to be 46,900 Da (PDI of 1.34) by GPC (using N,N-dimethylacetamide as eluent) against polystyrene standards.

### Quaternisation:

In a round bottom flask, the above polymer BC-6-1 (25 mg) was dissolved in acetonitrile (5 mL) and an excess of methyl iodide (1 mL) was added into this solution. The reaction was stirred at room temperature overnight in order to achieve the quaternisation of the tertiary amino groups of the DMAEMA groups incorporated into the triblock copolymers. After removal of solvent (acetonitrile) to dryness, the quaternised ABA triblock polymer was obtained and subjected to the final purification step of dialysis (molecular weight cut-off of 3500, Spectra Por, Spectrum Medical Industries, Inc., Houston, Tx) against Milli-Q water for 3 days. After dialysis, the water was removed from the aqueous polymer solution by use of a freeze dryer.

### Structure of BC-6-1

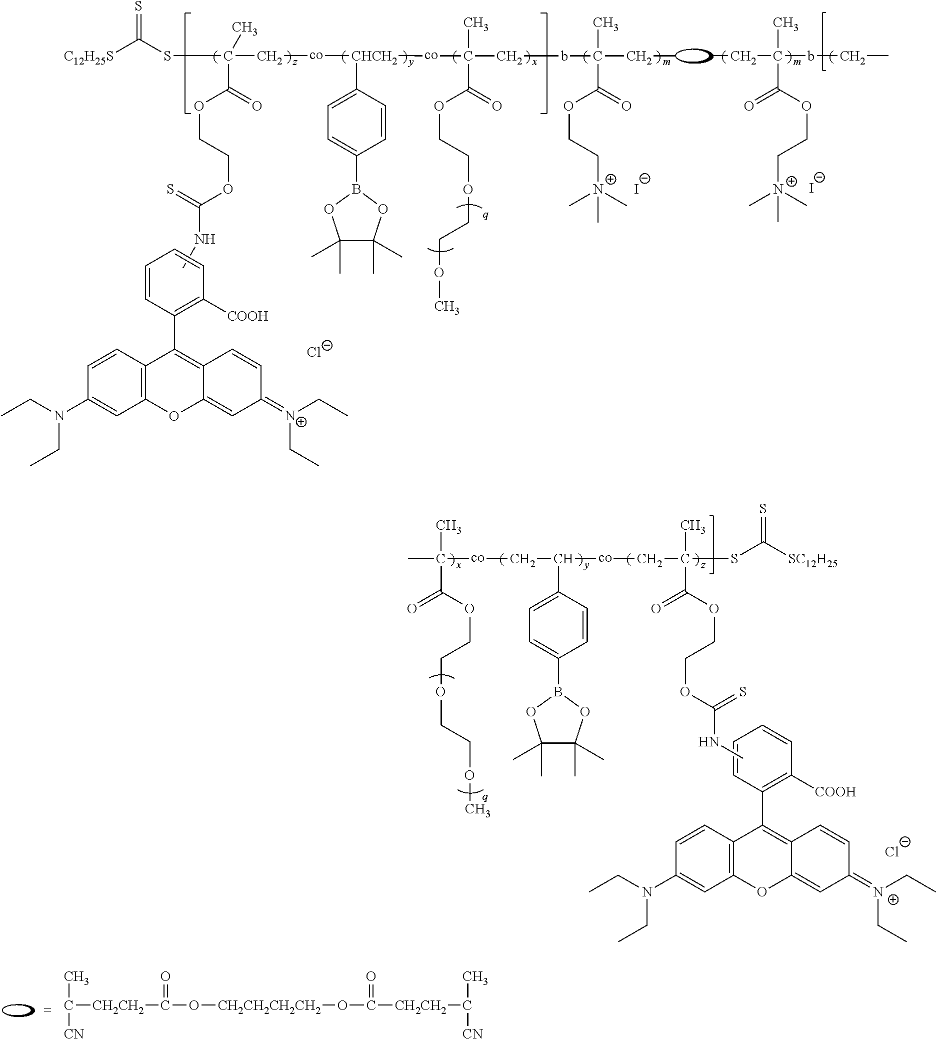

*4-vinylphenylboronic acid pinacol ester (VPBA-PE) was prepared from 4-vinylphenylboronic acid (VPBA) and pinacol in dichloromethane at room temperature in the presence of dried molecular sieves (4 Å).

Polymers BC-6-1 and BC-14 were evaluated for siRNA CHO-GFP silencing using the procedure described in Example 5. FIG. 15 illustrates the comparative silencing of CHO-GFP by the two polymer samples. Polymer sample with bound galactose (BC-14) exhibited significantly improved CHO-GFP silencing compared to the sample without galactose.

### Example 13

### Bis-RAFT Agent with Disulfide Linkage (III): (S)—(R)-11-cyano-11-methyl-8-oxo-13-thioxo-7-oxa-3,4,12,14-tetrathiahexacosyl 4-cyano-4-(((dodecylthio)carbonothioyl)thio)pentanoate

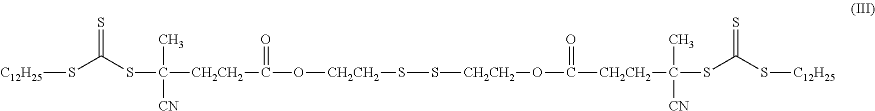

(S)-4-cyano-4-(dodecylthiocarbonothioylthio)pentanoic acid (8.06 g, 20.0 mmol), 2-hydroxyethyl disulfide (1.54 g, 10 mmol), DIC (diisopropylcarbodiimide, 2.77 g, 22.0 mmol) in dichloromethane (100 mL) and DMAP (N,N-dimethylaminopyridine, catalytic amount) were allowed to stir at room temperature for one hour. Filtered to remove the DIC-urea by-product and after removal of solvent, gave 9.2 g crude product which was purified by column chromatography on a silica-gel column using ethyl acetate:n-hexane 1:4 (v/v) as eluent to give the title product (III) (8.0 g, 86.5% yield) as a yellow oil. Proton nuclear magnetic resonance (¹H NMR) (CDCl₃) (ppm) 0.89 (t, 6H, 2×CH₃); 1.27 (br s, 36H); 1.69 (m, 4H); 1.88 (s, 6H, 2×CH₃); 2.40-2.80 (m, 8H, 2×CH₂CH₂); 2.90 (t, 4H, CH₂S—SCH₂); 3.30 (t, 4H, 2×CH₂S); 4.35 (t, 4H, 2×CH₂O).

### Linear ABA Triblock Copolymer ABA-B2S-53/55 (LN2012/1TL15)

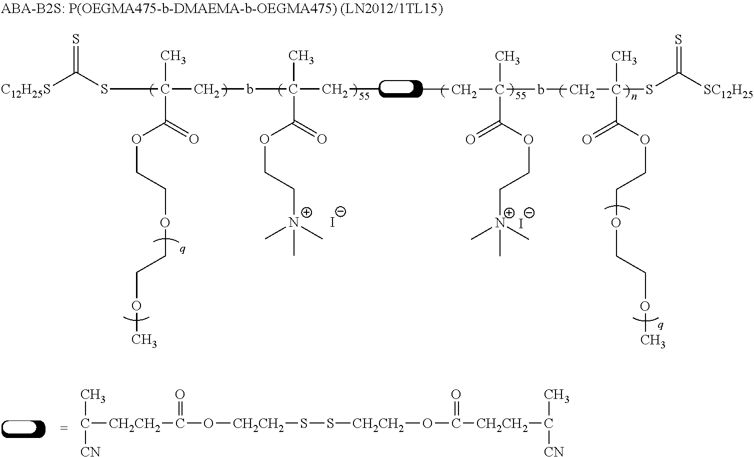

### Methods

### Step 1: Synthesis and characterization of poly(N,N-dimethylaminoethyl methacrylate) (PDMAEMA) telechelic macroRAFT agent

In a typical polymerization experiment, DMAEMA monomer (5.955 g, 3.788×10⁻² mol), VAZO-88 initiator (2.948×10⁻³ g, 1.207×10⁻⁵ mol); Bis-RAFT agent (III) (0.240 g, 2.413×10⁻⁴ mol) and DMF (26.8851 g, 3.678×10⁻¹ mol) were weighed into a Schlenk flask. The solution mixture was degassed with four freeze-evacuate-thaw cycles and polymerized at 90° C. for 17 hours.

The monomer to polymer conversion was 69.6% as determined by ¹H-NMR (in CDCl₃). The conversion was calculated by adding an internal standard 1,3,5-trioxane to the polymerization solution at an amount of 5 mg/1 mL. ¹H-NMR spectra before and after polymerization were compared; the integration of the —OCH₂ cyclic of the trioxane at 5.1 ppm was compared to that of the integration of the CH₂═C protons of the monomer at 5.5-6 ppm. The molecular weight of the polymer calculated based on ¹H-NMR was 17.2 kDa corresponds to a degree of polymerization of 109. The number average molecular weight (Mₙ) of the polymer as determined by gel permeation chromatography (GPC) against linear polystyrene standards was 22 kDa (dispersity of 1.23). Three different polymer samples were prepared by varying the block lengths of hydrophilic block (A) and the cationic block (B).

The polymers were quaternised using the procedure described in Example 1 and purified by dialysis.

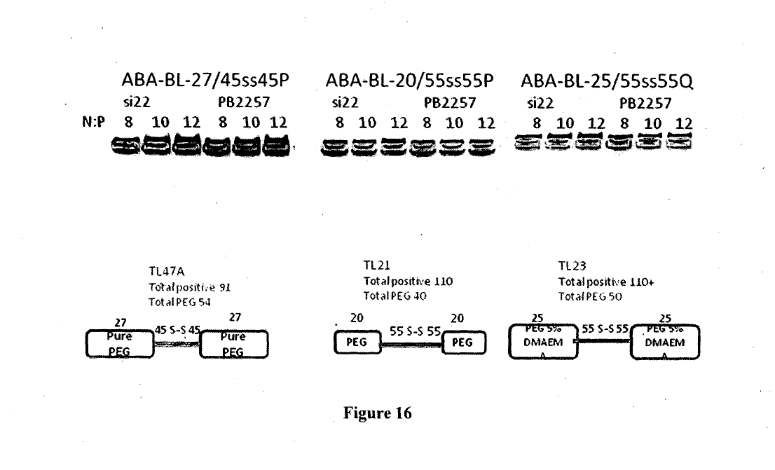

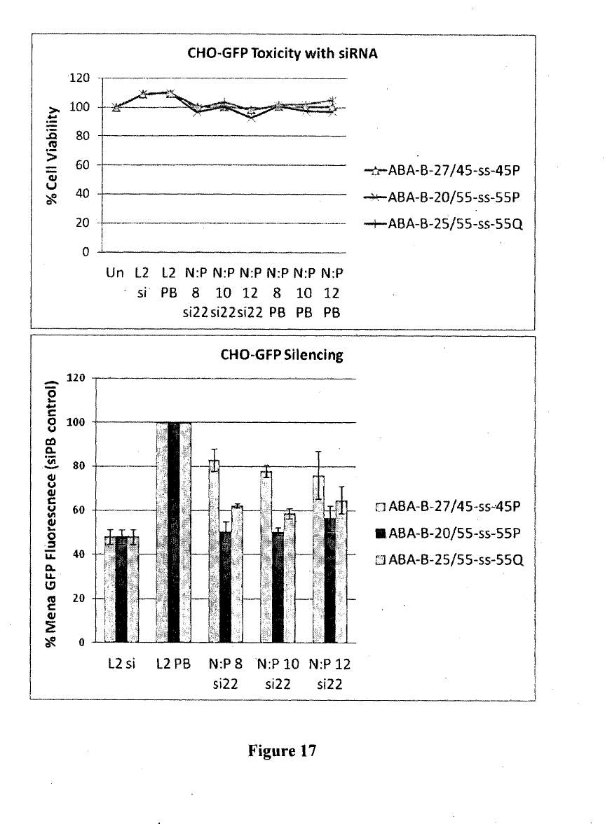

The dialysed polymer samples were evaluated for toxicity and silencing of CHO-GFP cells using the test methods described in Examples 2 and 5, respectively. The polymer/siRNA complex was stable at a range of molar ratios as illustrated in FIG. 16. The cell viability of siRNA/polymer complexes and CHO-GFP silencing of the polymer complexes are illustrated in FIG. 17, top and bottom panels, respectively.

1. A complex comprising a cationic block copolymer and a nucleic acid, the cationic block copolymer having at least a tri-block structure comprising a cationic block and two hydrophilic blocks, or a hydrophilic block and two cationic blocks.

2. The complex according to claim 1, wherein the at least tri-block structure of the cationic block copolymer is linear and comprises a cationic block and two hydrophilic blocks, and wherein the cationic block is located in between each of the two hydrophilic blocks.

3. The complex according to claim 2, wherein cationic block comprises about 40 to about 200 polymerised monomer residue units.

4. The complex according to claim 1, wherein the at least tri-block structure of the cationic block copolymer is linear and comprises a hydrophilic block and two cationic blocks, and wherein the hydrophilic block is located in between each of the two cationic blocks.

5. The complex according to claim 4, wherein each cationic block independently comprises about 20 to about 100 polymerised monomer residue units.

6. The complex according to claim 1, wherein the tri-block structure is a tri-block RAFT polymer structure.

7. The complex according to claim 1, wherein the cationic block comprises one more polymerised monomer residues selected from 2-Aminoethyl methacrylate hydrochloride, N-[3-(N,N-Dimethylamino)propyl]methacrylamide, N-(3-Aminopropyl)methacrylamide hydrochloride, N-[3-(N,N-Dimethylamino)propyl]acrylamide, N-[2-(N,N-Dimethylamino)ethyl]methacrylamide, 2-N-Morpholinoethyl acrylate, 2-N-Morpholinoethyl methacrylate, 2-(N,N-dimethylamino)ethyl acrylate, 2-(N,N-dimethylamino)ethyl methacrylate, 2-(N,N-diethylamino)ethyl methacrylate, 2-Acryloxyyethyltrimethylammonium chloride, Methacrylamidopropyltrimethylammonium chloride, 2-(tert-butylamino)ethyl methacrylate, Diallyldimethylammonium chloride, 2-(Diethylamino)ethylstyrene, 2-Vinylpyridine, and 4-Vinylpyridine.

8. The complex according to claim 1, wherein the nucleic acid is selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear RNAs (SnRNAs) ribozymes, aptamers, DNAzymes, ribonuclease complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

9. The complex according to claim 1 having a Zeta potential ranging from about 4 mV to about 40 mV.

10. A method of delivering a nucleic acid to a cell, the method comprising:
preparing a complex comprising a cationic block copolymer and a nucleic acid, the cationic block copolymer having at least a tri-block structure comprising a cationic block and two hydrophilic blocks, or a hydrophilic block and two cationic blocks; and introducing the complex to the cell.

11. A method of silencing gene expression, the method comprising transfecting a cell with a complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

12. A method of protecting a nucleic acid form enzymatic degradation, the method comprising complexing the nucleic acid with a cationic block copolymer, the cationic block copolymer having at least a tri-block structure comprising a cationic block and two hydrophilic blocks, or a hydrophilic block and two cationic blocks.

13. The method according to claim 10 when performed in vivo.

14. The method according to claim 10, wherein the at least tri-block structure of the cationic block copolymer is linear and comprises a cationic block and two hydrophilic blocks, and wherein the cationic block is located in between each of the two hydrophilic blocks.

15. The method according to claim 10, wherein the at least tri-block structure of the cationic block copolymer is linear and comprises a hydrophilic block and two cationic blocks, and wherein the hydrophilic block is located in between each of the two cationic blocks.

16. The method according to claim 10, wherein the tri-block structure is a tri-block RAFT polymer structure.

17. The method according to claim 10, wherein the cationic block comprises one more polymerised monomer residues selected from 2-Aminoethyl methacrylate hydrochloride, N-[3-(N,N-Dimethylamino)propyl]methacrylamide, N-(3-Aminopropyl)methacrylamide hydrochloride, N-[3-(N,N-Dimethylamino)propyl)acrylamide, N[2-(N,N-Dimethylamino)ethyl]methacrylamide, 2-N-Morpholinoethyl acrylate, 2-N-Morpholinoethyl methacrylate, 2-(N,N-dimethylamino)ethyl acrylate, 2-(N,N-dimethylamino)ethyl methacrylate, 2-(N,N-dimethylamino)ethyl methacrylate, 2-Acryloxyyethyltrimethyl ammonium chloride, Methacrylamidopropyltrimethylammonium chloride, 2-(tert-butylamino)ethyl methacrylate, Diallyldimethylammonium chloride, 2-(Diethylamino)ethylstyrene, 2Vinylpyridine, 4-Vinylpyridine

18. The method according to claim 10, wherein the nucleic acid is selected from gDNA, cDNA, double or single stranded DNA oligonucleotides, sense RNAs, antisense RNAs, mRNAs, tRNAs, rRNAs, small/short interfering RNAs (siRNAs), double-stranded RNAs (dsRNA), short hairpin RNAs (shRNAs), piwi-interacting RNAs (PiRNA), micro RNA/small temporal RNA (miRNA/stRNA), small nucleolar RNAs (SnoRNAs), small nuclear RNAs (SnRNAs) ribozymes, aptamers, DNAzymes, ribonuclease complexes, hairpin double stranded RNA (hairpin dsRNA), miRNAs which mediate spatial development (sdRNAs), stress response RNA (srRNAs), cell cycle RNA (ccRNAs) and double or single stranded RNA oligonucleotides.

19. The method according to claim 10, wherein said complex has a Zeta potential ranging from about 4 mV to about 40 mV.

20. The method according to claim 11, wherein the nucleic acid is capable of silencing the expression of a virus derived gene in the cell.

21. Use of a complex for delivering a nucleic acid to a cell, the complex comprising a cationic block copolymer and the nucleic acid, the cationic block copolymer having at least a tri-block structure comprising a cationic block and two hydrophilic blocks, or a hydrophilic block and two cationic blocks.

22. Use of a complex for silencing gene expression, the complex comprising a cationic block copolymer and a nucleic acid selected from DNA and RNA, the cationic block copolymer having at least a tri-block structure comprising a cationic block and two hydrophilic blocks, or a hydrophilic block and two cationic blocks.

23. Use of a cationic block copolymer in protecting a nucleic acid from enzymatic degradation, the cationic block copolymer having at least a tri-block structure comprising a cationic block and two hydrophilic blocks, or a hydrophilic block and two cationic blocks.


SEQUENCE LISTING (2 sequences)

SEQ ID NO: 1 - DNA, 22 bp, Artificial
  Synthetic
gcaagcugacccugaaguucau

SEQ ID NO: 2 - DNA, 22 bp, Artificial
  Synthetic
gaacuucagggucagcuugccg